(19) United States
(12) Patent Application Publication
(10) Pub. No.: US 2022/0110673 A1
(43) Pub. Date: Apr. 14, 2022

(54) STRUCTURED TISSUE CONTACT SURFACE FOR ENERGY-BASED SURGICAL INSTRUMENT

(71) Applicant: Cilag GmbH International, Zug (CH)

(72) Inventors: Steven M. Boronyak, Cincinnati, OH (US); John M. Sarley, Mason, OH (US); Donald L. Reynolds, II, West Chester, OH (US); Michael A. Keenan, Cincinnati, OH (US); Duan Li Ou, Warren, NJ (US)

(21) Appl. No.: 17/472,749

(22) Filed: Sep. 13, 2021

Related U.S. Application Data
(60) Provisional application No. 63/090,749, filed on Oct. 13, 2020.

(51) Int. Cl. A61B 18/14; A61B 17/32; B08B 7/00

(52) U.S. Cl. CPC A61B 18/1445; A61B 2017/00938; B08B 7/0035; A61B 17/320092

(57) ABSTRACT

A method of manufacturing a surgical instrument that includes an energized feature operable to apply ultrasonic energy or RF energy to tissue. The method includes forming at least one of a microscopic surface pattern or a nanoscopic surface roughness into a base surface of the energized feature to produce at least one recessed portion. The method also includes applying a hydrophobic coating that includes at least one of silicone, titanium nitride, chromium nitride, or titanium aluminum nitride to at least the recessed portion of the energized feature after forming at least one of the microscopic surface pattern or the nanoscopic surface roughness.

### PRIORITY

This application claims priority to U.S. Provisional Patent Application No. 63/090,749, entitled “Structured Tissue Contact Surface for Energy-Based Surgical Instrument,” filed on Oct. 13, 2020, the disclosure of which is hereby incorporated by reference herein.

### BACKGROUND

A variety of ultrasonic surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned, the disclosure of which is incorporated by reference herein; U.S. Pub. No. 2007/0191713, entitled “Ultrasonic Device for Cutting and Coagulating,” published Aug. 16, 2007, now abandoned, the disclosure of which is incorporated by reference herein; and U.S. Pub. No. 2008/0200940, entitled “Ultrasonic Device for Cutting and Coagulating,” published Aug. 21, 2008, now abandoned, the disclosure of which is incorporated by reference herein.

Some instruments are operable to seal tissue by applying radiofrequency (RF) electrosurgical energy to the tissue. Examples of such devices and related concepts are disclosed in U.S. Pat. No. 7,354,440, entitled “Electrosurgical Instrument and Method of Use,” issued Apr. 8, 2008, the disclosure of which is incorporated by reference herein; U.S. Pat. No. 7,381,209, entitled “Electrosurgical Instrument,” issued Jun. 3, 2008, the disclosure of which is incorporated by reference herein.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein; and U.S. Pat. No. 8,663,220, entitled “Ultrasonic Electrosurgical Instruments,” issued Mar. 4, 2014, the disclosure of which is incorporated by reference herein.

U.S. Pat. No. 9,272,095, entitled “Vessels, Contact Surfaces, and Coating and Inspection Apparatus and Methods,” issued on Mar. 1, 2016 relates to fabrication of coated contact surfaces of a medical device. U.S. Pat. No. 9,272,095 describes one utility for such a hydrophobic layer is to isolate a thermoplastic tube wall, made for example of polyethylene terephthalate (PET), from blood collected within the tube. A hydrophobic layer can be applied on top of a hydrophilic SiO, coating on the internal contact surface of the tube and the hydrophobic layer precursor can comprise hexamethyldisiloxane (HMDSO) or octamethylcyclotetrasiloxane (OMCTS). U.S. Pat. No. 9,272,095 does not appear to disclose hydrophobic coating being applied in addition to at least one of the microscopic surface pattern or the nanoscopic surface roughness.

U.S. Pub. No. 2014/0276407, entitled “Medical Devices Having Micropatterns,” published on Sep. 14, 2014, now abandoned, describes a plurality of nanostructures, a plurality of microstructures, and a plurality of hierarchical structures. A micropatterned polymer coating may be formed of any suitable material for a particular application, and may include one or more of a flexible polymer, a rigid polymer, a metal, an alloy, and any other material that may be suitable for a particular application. The micropatterned polymer coating could be applied by any of a wide variety of manufacturing techniques described herein including extrusion, compression dies, electro deposition, photoetching, or over molding configurations. U.S. Pub. No. 2014/0276407 does not appear to disclose a hydrophobic coating being applied in addition to at least one of the microscopic surface pattern or the nanoscopic surface roughness.

U.S. Pub. No. 2013/0138103 entitled “Electrosurgical Unit with Micro/nano Structure and the Manufacturing Method Thereof,” published on May 30, 2013, now abandoned, describes in FIG. 2 using the irradiation of the laser beam to construct directly a micro/nano structure on the surface of the blade while allowing the micro/nano structure to be composed of a hybrid of micro/nano elements. Referring to FIG. 3, the micro/nano structure 13 is formed directly on the blade 11. U.S. Pub. No. 2013/0138103 does not appear to disclose a hydrophobic coating in addition to the micro/nano structure.

U.S. Pat. No. 9,434,857, entitled “Rapid Cure Silicone Lubricious Coatings,” issued Sep. 6, 2016 describes lubricious silicone coating compositions which are particularly useful for coating surfaces of medical devices such as surgical needles and other tissue piercing or cutting devices. The compositions include a mixture of a cross-linkable siloxane polymer and a non-cross-linkable siloxane polymer, a conventional silicone cross-linking agent, and a platinum catalyst. The silicone polymer components are blended with conventional aromatic organic solvents, including, for example, xylene and aliphatic organic solvents (such as, for example, hexane or its commercial derivatives) to form coating solutions or compositions. U.S. Pat. No. 9,434,857 does not appear to disclose a hydrophobic coating being applied in addition to at least one of the microscopic surface pattern or the nanoscopic surface roughness.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side elevational view of an exemplary ultrasonic surgical instrument;

FIG. 2 depicts a side elevational view of an end effector of the instrument of FIG. 1, with the end effector including an energized feature in the form of an ultrasonic blade;

FIG. 3 depicts a perspective view of an exemplary radiofrequency electrosurgical instrument;

FIG. 4 depicts an enlarged perspective view of an exemplary articulation assembly and an end effector of the instrument of FIG. 3, with the end effector including an energized feature in the form of a pair of electrodes;

FIG. 5 depicts a perspective view of a first exemplary tissue release feature in the form of a microscopic surface pattern applied to the energized feature of FIG. 2;

FIG. 6 depicts a cross-sectional view of the microscopic surface pattern of FIG. 5 taken along line 6-6 of FIG. 5;

FIG. 7 depicts a perspective view of a second exemplary tissue release feature in the form of a first microscopic surface pattern applied to the energized feature of FIG. 2;

FIG. 8 depicts a cross-sectional view of the microscopic surface pattern of FIG. 7 taken along line 8-8 of FIG. 7;

FIG. 9 depicts a perspective view of a third exemplary tissue release feature in the form of a second microscopic surface pattern applied to the energized feature of FIG. 2;

FIG. 10 depicts a cross-sectional view of the microscopic surface pattern of FIG. 9 taken along line 8-8 of FIG. 9;

FIG. 11 depicts a perspective view of a fourth exemplary tissue release feature in the form of a third microscopic surface pattern applied to the energized feature of FIG. 2;

FIG. 12 depicts a cross-sectional view of the microscopic surface pattern of FIG. 11 taken along line 12-12 of FIG. 11;

FIG. 13 depicts a perspective view of a second exemplary tissue release feature in the form of a fourth microscopic surface pattern applied to the energized feature of FIG. 2;

FIG. 14 depicts a cross-sectional view of the microscopic surface pattern of FIG. 13 taken along line 14-14 of FIG. 13;

FIG. 15 depicts a perspective view of a second exemplary tissue release feature in the form of a fifth microscopic surface pattern applied to the energized feature of FIG. 2;

FIG. 16 depicts a cross-sectional view of the microscopic surface pattern of FIG. 15 taken along line 16-16 of FIG. 15;

FIG. 17 depicts a line graph showing plots of the sticking force versus the run order of the cross-groove pattern of FIG. 6, of the array of dimples of FIG. 13, and of a flat non-patterned surface;

FIG. 18 depicts a box plot graph showing plots of the number of cleanly releasing activations for a flat non-patterned surface and the cross-groove pattern of FIG. 6;

FIG. 19 depicts a box plot graph showing plots of the sticking force of the hydrophobic coated cross-groove pattern of FIG. 6, a non-coated cross-groove pattern similar to FIG. 6, and of a flat non-coated non-patterned surface;

FIG. 20A depicts a schematic cross-sectional view of a third exemplary tissue release feature in the form of nanoscopic surface roughness that includes a hydrophobic coating applied to the energized feature of FIG. 2, prior to a portion of the hydrophobic coating being worn away;

FIG. 20B depicts a schematic cross-sectional view of the nanoscopic surface roughness of FIG. 20A, after a portion of the hydrophobic coating is worn away;

FIG. 21A depicts a schematic view of an exemplary tissue release feature in the form of an exemplary hierarchical surface structure that includes a microscopic surface pattern and a nanoscopic surface roughness with a hydrophobic coating applied to the energized feature of FIG. 2, prior to a portion of the hydrophobic coating being worn away;

FIG. 21B depicts a schematic cross-sectional view of the hierarchical surface structure of FIG. 20A, after a portion of a hydrophobic coating is worn away;

FIG. 22A depicts an enlarged cross-sectional view of the hierarchical surface structure of FIG. 21A, prior to a portion of the hydrophobic coating being worn away;

FIG. 22B depicts an enlarged cross-sectional view of the hierarchical surface structure of FIG. 21B, after a portion of the hydrophobic coating is worn away;

FIG. 23 depicts a diagrammatic view of a first exemplary method of manufacturing the energized feature of FIG. 2;

FIG. 24 depicts a diagrammatic view of a second exemplary method of applying a hydrophobic coating to the energized feature of FIG. 2;

FIG. 25 depicts a diagrammatic view of a third exemplary method of applying a hydrophobic coating to the energized feature of FIG. 2; and

FIG. 26 depicts a diagrammatic view of a fourth exemplary method of applying a hydrophobic coating to the energized feature of FIG. 2.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” and “bottom,” are used with respect to the examples and associated figures and are not intended to unnecessarily limit the invention described herein.

I. Exemplary Ultrasonic Surgical Instrument with Integrated RF Energy

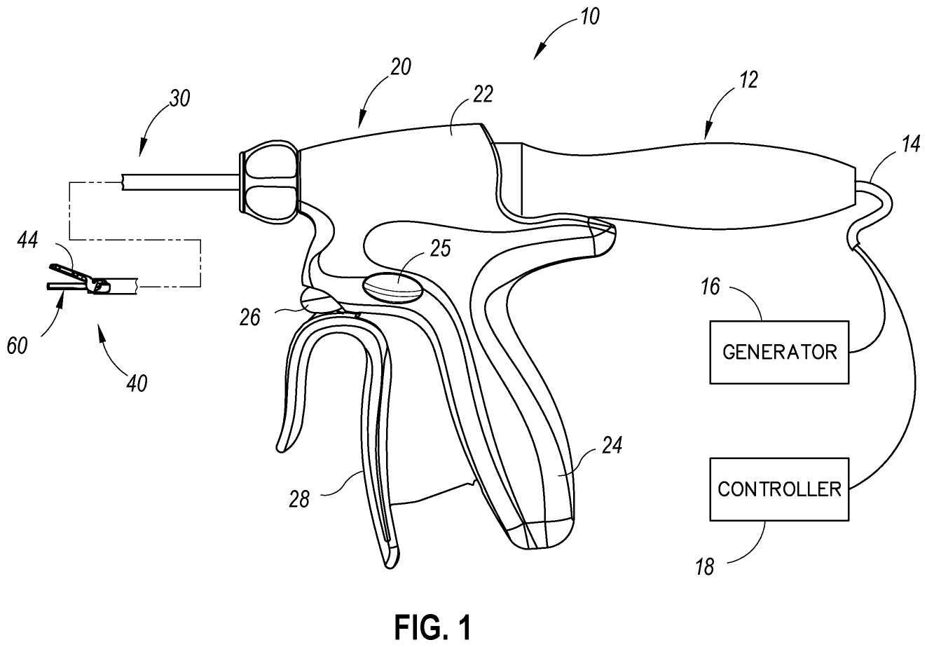

FIG. 1 illustrates an exemplary ultrasonic surgical instrument (10). At least part of instrument (10) may be constructed and operable in accordance with at least some of the teachings of any of the patent references that are cited herein. Instrument (10) is operable to cut tissue and seal or weld tissue (e.g., a blood vessel, etc.) substantially simultaneously.

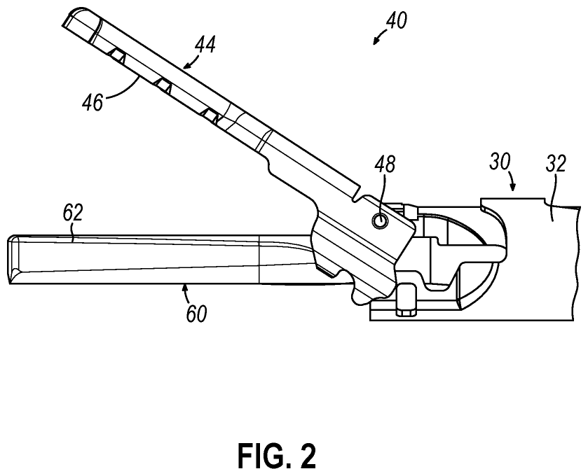

Instrument (10) of the present example comprises a handle assembly (20), a shaft assembly (30), and an end effector (40). Handle assembly (20) comprises a body (22) including a pistol grip (24) and a pair of buttons (25, 26). Handle assembly (20) includes a trigger (28) that is pivotable toward and away from pistol grip (24). It should be understood, however, that various other suitable configurations may be used, including but not limited to a scissor grip configuration. As best seen in FIG. 2, end effector (40) includes an energized feature (shown as an ultrasonic blade (60)) and a pivoting clamp arm (44). Clamp arm (44) is coupled with trigger (28) such that clamp arm (44) is pivotable toward ultrasonic blade (60) in response to pivoting of trigger (28) toward pistol grip (24). Clamp arm (44) is pivotable away from ultrasonic blade (60) in response to pivoting of trigger (28) away from pistol grip (24). Buttons (25, 26) may provide the operator with varied control of the energy that is applied to tissue through end effector (40). For instance, buttons (25, 26) may provide functionality in accordance with at least some of the teachings of U.S. Pat. No. 9,949,785, entitled “Ultrasonic Surgical Instrument with Electrosurgical Feature,” issued Apr. 24, 2018, the disclosure of which is incorporated by reference herein.

An ultrasonic transducer assembly (12) extends proximally from body (22) of handle assembly (20) in the present example. Transducer assembly (12) is coupled with a generator (16) via a cable (14). Transducer assembly (12) receives electrical power from generator (16) and converts that electrical power into ultrasonic vibrations through piezoelectric principles as is known in the art. Generator (16) cooperates with a controller (18) to provide a power profile to transducer assembly (12) that is particularly suited for the generation of ultrasonic vibrations through transducer assembly (12). In addition, or in the alternative, generator (16) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein.

As shown, ultrasonic blade (60) includes an outer surface (62). Clamp arm (44) includes a clamp pad that is secured to the underside of clamp arm (44), facing blade (60). By way of further example only, the clamp pad may be further constructed and operable in accordance with at least some of the teachings of U.S. Pat. No. 7,544,200, entitled “Combination Tissue Pad for Use with an Ultrasonic Surgical Instrument,” issued Jun. 9, 2009, the disclosure of which is incorporated by reference herein. Clamp arm (44) is operable to selectively pivot toward and away from ultrasonic blade (60) about a pivot pin (48) to selectively clamp tissue between clamp arm (44) and ultrasonic blade (60) in response to pivoting of trigger (28) toward pistol grip (24).

Ultrasonic blade (60) of the present example is operable to vibrate at ultrasonic frequencies to effectively cut through and seal tissue, particularly when the tissue is being clamped between clamp arm (44) and ultrasonic blade (60). Ultrasonic blade (60) is positioned at the distal end of an acoustic drivetrain that includes an acoustic waveguide (not shown) and transducer assembly (12) to vibrate ultrasonic blade (60). Ultrasonic blade (60) is in acoustic communication with the acoustic waveguide. By way of further example only, the acoustic waveguide and ultrasonic blade (60) may be constructed and operable in accordance with the teachings of U.S. Pat. No. 6,423,082, entitled “Ultrasonic Surgical Blade with Improved Cutting and Coagulation Features,” issued Jul. 23, 2002, the disclosure of which is incorporated by reference herein.

In the present example, the distal end of ultrasonic blade (60) is located at a position corresponding to an anti-node associated with resonant ultrasonic vibrations communicated through a flexible acoustic waveguide, to tune the acoustic assembly to a preferred resonant frequency fₒ when the acoustic assembly is not loaded by tissue. When transducer assembly (12) is energized, the distal end of ultrasonic blade (60) is configured to move longitudinally in the range of, for example, about 10 to 500 microns peak-to-peak, and in some instances in the range of about 20 to about 200 microns at a predetermined vibratory frequency fₒ of, for example, 50 kHz or 55.5 kHz. When transducer assembly (12) of the present example is activated, these mechanical oscillations are transmitted through waveguides to reach blade (60), thereby providing oscillation of ultrasonic blade (60) at the resonant ultrasonic frequency. Thus, when tissue is secured between ultrasonic blade (60) and clamp arm (44), the ultrasonic oscillation of blade (60) may simultaneously sever the tissue and denature the proteins in adjacent tissue cells, thereby providing a coagulative effect with relatively little thermal spread.

In some versions, end effector (40) may be configured to apply radiofrequency (RF) electrosurgical energy to tissue that is captured between clamp arm (44) and ultrasonic blade (60). By way of example only, clamp arm (44) may include one or more RF electrodes and/or ultrasonic blade (60) may serve as an RF electrode. In such versions, the control of ultrasonic energy and RF electrosurgical energy may be provided in accordance with at least some of the teachings of U.S. Pat. No. 8,663,220, entitled “Ultrasonic Electrosurgical Instruments,” issued Mar. 4, 2014, the disclosure of which is incorporated by reference herein; and/or U.S. Pat. No. 9,949,785, entitled “Ultrasonic Surgical Instrument with Electrosurgical Feature,” issued Apr. 24, 2018, the disclosure of which is incorporated by reference herein.

II. Exemplary Radiofrequency Surgical Instrument

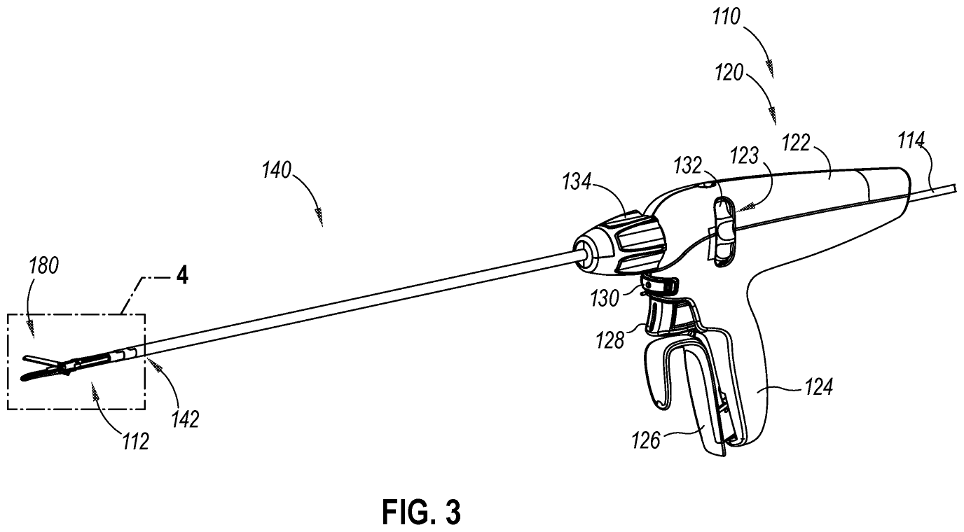

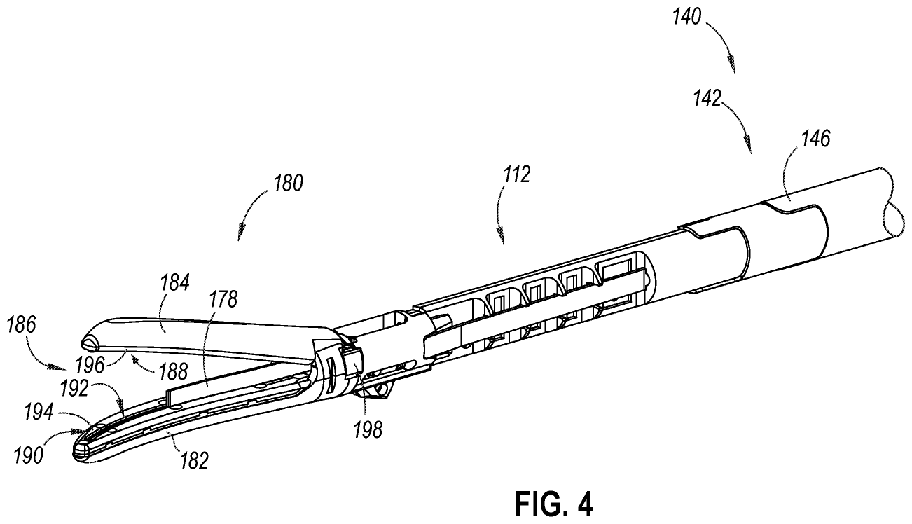

FIGS. 3-4 show an exemplary electrosurgical instrument (110). As best seen in FIG. 3, instrument (110) includes a handle assembly (120), a shaft assembly (140), an articulation assembly (112), and an end effector (180). Shaft assembly (140) extends distally from handle assembly (120) and connects with articulation assembly (112). End effector (180) extends distally from shaft assembly (140) and is operable to grasp, cut, and seal or weld tissue (e.g., a blood vessel, etc.). In this example, end effector (180) is configured to seal or weld tissue by applying bipolar radiofrequency (RF) energy to tissue. In the present example, electrosurgical instrument (110) is electrically coupled to a power source (not shown) via power cable (114). The power source may be configured to provide all or some of the electrical power requirements for use of instrument (110). By way of example only, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein.

Handle assembly (120) includes a body (122), a pistol grip (124), a jaw closure trigger (126), a knife trigger (128), an activation button (130), an articulation control (132), and a knob (134). Jaw closure trigger (126) may be pivoted toward and away from pistol grip (124) and/or body (122) to open and close jaws (182, 184) of end effector (180) to grasp tissue. Knife trigger (128) may be pivoted toward and away from pistol grip (124) and/or body (122) to actuate a knife member (178) within the confines of jaws (182, 184) to cut tissue captured between jaws (182, 184). Activation button (130) may be pressed to apply radio frequency (RF) energy to tissue via electrode surfaces (194, 196) of jaws (182, 184), respectively. Knob (134) is rotatably disposed on the distal end of body (122) and is configured to rotate end effector (180), articulation assembly (112), and shaft assembly (140) about the longitudinal axis of shaft assembly (140) relative to handle assembly (120).

FIG. 4 shows articulation assembly (112), a distal portion (142) of shaft assembly (140), and end effector (180). Articulation assembly (112) is connected with a proximal end of end effector (180). Articulation assembly (112) is configured to deflect end effector (180) from the longitudinal axis defined by shaft assembly (140). As best seen in FIG. 4, end effector (180) includes lower jaw (182) pivotally coupled with an upper jaw (184) via pivot couplings (198). Lower jaw (182) includes a proximal body (183). Slots (186, 188) each slidably receive pin (not shown). Upper jaw (184) is configured to pivot toward and away from lower jaw (182) about pivot couplings (198) to grasp tissue.

End effector (180) includes an energized feature (shown as electrode assembly (186)) that is configured to apply energy to treat tissue. Electrode assembly (186) includes electrodes (188, 190). Electrodes (188, 190) are configured to cooperate to apply bipolar RF energy to tissue. Upper jaw (184) is shown as a clamp arm that is configured to compress tissue against electrode assembly (186). As shown, electrode (188) includes electrode surface (194), and electrode (190) includes electrode surface (196). Lower jaw (182) and upper jaw (184) each comprise a respective electrode surface (194, 196). The power source may provide RF energy to electrode surfaces (194, 196) via electrical wire (not shown) that extends through handle assembly (120), shaft assembly (140), articulation assembly (112), and electrically couples with one or both of electrode surfaces (194, 196). An electrical wire (not shown) may selectively activate electrode surfaces (194, 196) in response to an operator pressing activation button (130). By way of example only, end effector (40) may include a single “active” electrode (e.g., one of electrodes (188, 190)) that cooperates with a conventional ground pad that is secured to the patient, such that end effector (40) applies monopolar RF electrosurgical energy to the tissue. Lower jaw (182) and upper jaw (184) define a knife pathway (192). Knife pathway (192) is configured to slidingly receive knife member (178), such that knife member (178) may be retracted and advanced to cut tissue captured between jaws (182, 184).

III. Exemplary Tissue Release Features

### A. Overview

Instruments (10, 110) may generate heat as end effectors (40, 180) seal and/or cut tissue. Energized features may tend to stick to the treated tissue at a contact interface, where the energized feature and the tissue contact one another. The energized feature is intended to include at least one of ultrasonic blade (60) shown in FIGS. 1-2, electrodes (188, 190) of electrode assembly (186)) shown in FIG. 4, or another suitable energized feature. The energized feature includes a base surface that is configured to contact the tissue. For example, base surfaces may include, for example, outer surface (62) of ultrasonic blade (60), electrode surface (194) of electrode (188), and/or electrode surface (196) of electrode (190). Tissue sticking may cause reduced surgical efficiency. While a hydrophobic coating may be applied to flat surfaces of the energized feature to help reduce tissue sticking, the hydrophobic coating may prematurely wear away over time from the flat surface with increased instrument use. For example, a non-durable hydrophobic coating may wear away from the flat surface over the course of a single procedure. As a result, it may be desirable to reduce, or altogether eliminate, tissue sticking without experiencing problems that may otherwise be associated with a hydrophobic coating applied to the flat surface.

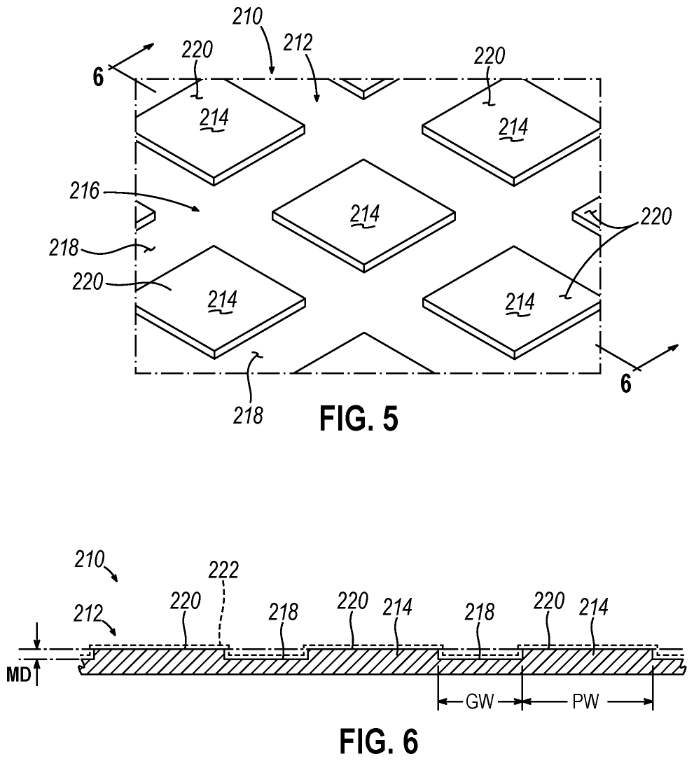

As will be described in greater detail below with reference to FIGS. 5-20B, energized features (e.g., ultrasonic blade (60) and electrodes (188, 190) of electrode assembly (186)) may include one or more exemplary tissue release features (210, 310, 410, 510, 610, 710, 910, 1010) to reduce tissue sticking or otherwise promote tissue release. While tissue release features (210, 310, 410, 510, 610, 710, 910, 1010) are described with reference to being applied to ultrasonic blade (60) of FIGS. 1-2, tissue release features (210, 310, 410, 510, 610, 710, 910, 1010) may also be applied to at least one of electrode surfaces (196, 198) of electrodes (188, 190) or another suitable energized feature. As previously described, electrodes (188, 190) may be configured to cooperate to apply bipolar RF energy to tissue.

It is envisioned that tissue release features (210, 310, 410, 510, 610, 710, 910, 1010) may be applied to select portions of the energized features. Alternatively, tissue release features (210, 310, 410, 510, 610, 710, 910, 1010) may be applied to the entire energized feature. In some versions, tissue release feature (210, 310, 410, 510, 610, 710, 910, 1010) may be applied to the entire outer surface of ultrasonic blade (60) and electrodes (188, 190) of electrode assembly (186). In other versions, tissue release features (210, 310, 410, 510, 610, 710, 910, 1010) may be applied to only select outer surfaces or to select portions of select outer surfaces of ultrasonic blade (60) and electrodes (188, 190) of electrode assembly (186) that experience sticking or high-pressure during tissue clamping. Tissue release feature (210, 310, 410, 510, 610, 710, 910, 1010) may be disposed on a metallic surface of the energized feature. As will be described in greater detail below, tissue release features (210, 310, 410, 510, 610, 710, 910, 1010) may include a microscopic surface pattern (212, 312, 412, 512, 612, 712), a nanoscopic surface roughness (912), or a hierarchical surface structure (1012) that includes a combination of microscopic surface pattern (1014) and nanoscopic surface roughness (1016).

### B. Microscopic Surface Patterns

FIGS. 5-16 show exemplary tissue release features (210, 310, 410, 510, 610, 710) including exemplary microscopic surface patterns (212, 312, 412, 512, 612, 712) that provide a reduction in tissue sticking. Microscopic surface patterns (212, 312, 412, 512, 612, 712) may be more robust than hydrophobic coatings alone, and may be maintained over the life of instrument (10, 110). Microscopic surface patterns (212, 312, 412, 512, 612, 712) may include an optional hydrophobic coating (222, 322, 422, 522, 624, 724). Microscopic surface patterns (212, 312, 412, 512, 612, 712) may be formed in a base surface (214, 314, 414, 514, 614, 714) of ultrasonic blade (60) and/or electrodes (188, 190) of electrode assembly (186) that are used to seal and/or cut tissue. By controlling the size and depth of recessed portions relative to base surface (214, 314, 414, 514, 614, 714), microscopic surface patterns (212, 312, 412, 512, 612, 712) may decrease the amount of tissue sticking compared to base surfaces having generally smooth surfaces. For example, microscopic surface patterns (212, 312, 412, 512, 612, 712) disposed on metallic surfaces may reduce tissue sticking compared to smooth metallic surfaces, which may reduce the number of protein bonding sites.

As will be described in greater detail below with reference to FIGS. 5-16, microscopic surface patterns (212, 312, 412, 512, 612, 712) respectively include a plurality of recessed portions (216, 316, 416, 516, 616, 716) that are recessed at a microscopic depth (MD) from base surface (214, 314, 414, 514, 614, 714). Microscopic surface patterns (212, 312, 412, 512, 612, 712) may be formed using a subtractive manufacturing process (e.g., laser ablation or chemical etching). Base surfaces (214, 314, 414, 514, 614, 714) may remain following a subtractive manufacturing process (e.g., laser ablation or chemical etching). For example, a nanosecond laser may be used to ablate away material from base surface (214, 314, 414, 514, 614, 714) to produce microscopic surface patterns (212, 312, 412, 512, 612, 712). However, it is also envisioned that microscopic surface pattern (212, 312, 412, 512, 612, 712) may be formed using additive manufacturing. The microscopic scale (or microscale) refers to surface roughness with a length scale applicable to microtechnology, which may be cited as 1-100 micrometers (i.e., microns). To reduce tissue sticking, the microscopic depth (MD) of recessed portions (216) may be between approximately 5 microns and approximately 100 microns, or more particularly between approximately 7 microns and approximately 25 microns.

Microscopic surface patterns (212, 312, 412, 512, 612, 712) may reduce tissue sticking through at least two mechanisms. First, microscopic surface patterns (212, 312, 412, 512, 612, 712) may reduce tissue sticking to promote tissue release from the energized feature by increasing the hydrophobicity of the base surface, which increases the fluid contact angle. The fluid contact angle is the angle that a liquid forms when disposed on a substrate (e.g., an energized feature). Increasing the fluid contact angle increases the hydrophobicity and/or the oleophobicity of the contact surface. The fluid contact angle may be used to measure the wettability of a surface or material. Wettability generally refers to how the liquid spreads out when deposited on the substrate. When the surface is already hydrophobic (i.e., having a fluid contact angle greater than 90 degrees), such as a flat stainless steel electrode with a hydrophobic coating, then a similar surface that is a micropatterned stainless steel electrode with a hydrophobic coating applied on top may be more hydrophobic. However, flat stainless steel without a coating may be hydrophilic, and a micropatterned stainless steel electrode without a coating may be more hydrophilic than a flat one. In other words, microscopic surface patterns (212, 312, 412, 512, 612, 712) may amplify the effect (flat hydrophobic surfaces become more hydrophobic with a microstructure, flat hydrophilic surfaces likewise become more hydrophilic with a pattern). This may be mathematically seen by the Wenzel equation. Second, microscopic surface patterns (212, 312, 412, 512, 612, 712) may aid in tissue release by decreasing the surface area in direct, and relatively high pressure, contact with the tissue. For example, micropatterned stainless steel electrodes without coatings may experience less tissue sticking than flat stainless-steel electrodes without coatings. Decreasing the surface area in direct contact with the tissue may reduce tissue sticking because of a lower number of tissue bonding sites (e.g., protein bonding sites).

### 1. First Exemplary Microscopic Surface Pattern

FIGS. 5-6 show a first exemplary tissue release feature (210) including a first exemplary microscopic surface pattern (212) in the form of a cross-groove pattern applied to base surface (214) ultrasonic blade (60) of FIG. 2. Particularly, FIG. 5 shows a perspective view of microscopic surface pattern (212), and FIG. 6 shows a cross-sectional view of microscopic surface pattern (212) of FIG. 5 taken along line 6-6 of FIG. 5. Particularly, microscopic surface pattern (212) of tissue release feature (210) includes a plurality of grooves (218) and a plurality of rectangular pillars (220). As shown, grooves (218) intersect rectangular pillars (220) at approximately 90-degree angles. However, grooves (218) may intersect rectangular pillars (220) at variety of other suitable angles. As shown in FIG. 6, grooves (218) are recessed relative to rectangular pillars (220). An optional hydrophobic coating (222) may be applied to tissue release feature (210) to reduce tissue sticking. As shown, hydrophobic coating (222) has a thickness that may exceed 100 nanometers. In some versions, hydrophobic coating (222) completely fills grooves (218); yet in other versions, hydrophobic coating (222) has a thickness that is less than the microscopic depth (MD) of grooves (218).

In some versions, the microscopic depth (MD) of grooves (218) relative to rectangular pillars (220) may range from between approximately 5 microns and approximately 50 microns. Rectangular pillars (220) may have a groove width (GW) of between approximately 20 microns and approximately 150 microns. Rectangular pillars (220) may have a pillar width (PW) of between approximately 20 microns and approximately 200 microns. As shown, rectangular pillars (220) have a width of approximately 140 microns and a length of approximately 140 microns, which are separated by a grid of grooves (218) having a groove width (GW) of approximately 96 microns. As an additional example, microscopic surface pattern (212) may include rectangular pillars (220) having a width of approximately 51 microns and a length of approximately 51 microns, which are separated by a grid of grooves (218) having a width of approximately 43 microns. While rectangular pillars (220) are shown as being square shaped, a variety of other shapes for rectangular pillars (220) are also envisioned. Additionally, the arrangement of rectangular pillars (220) may be non-uniform.

### 2. Second Exemplary Microscopic Surface Pattern

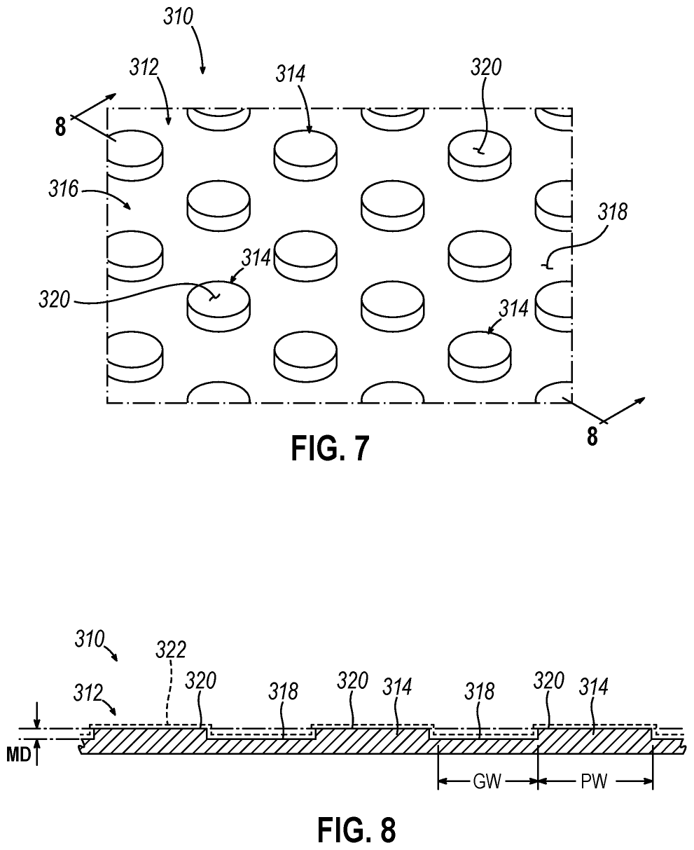

FIGS. 7-8 show a second exemplary tissue release feature (310) including a second exemplary microscopic surface pattern (312) in the form of a cross-groove pattern applied to base surface (314) ultrasonic blade (60) of FIG. 2. Particularly, FIG. 7 shows a perspective view of microscopic surface pattern (312), and FIG. 8 shows a cross-sectional view of microscopic surface pattern (312) of FIG. 8 taken along line 8-8 of FIG. 7. Particularly, microscopic surface pattern (312) of tissue release feature (310) includes a plurality of grooves (318) and a plurality of circular pillars (320). As shown, grooves (318) intersect circular pillars (320) at approximately 90-degree angles. However, grooves (318) may intersect circular pillars (320) at variety of other suitable angles. As shown in FIG. 8, grooves (318) are recessed relative to circular pillars (320). An optional hydrophobic coating (322) may be applied to tissue release feature (310) to reduce tissue sticking. As shown, hydrophobic coating (322) has a thickness that is less than the microscopic depth (MD) of grooves (318).

In some versions, the microscopic depth (MD) of grooves (318) relative to circular pillars (320) may range from between approximately 5 microns and approximately 50 microns. Circular pillars (320) may have a groove width (GW) of between approximately 20 microns and approximately 150 microns. Circular pillars (320) may have a pillar width (PW), also considered a pillar diameter, of between approximately 20 microns and approximately 200 microns. Additionally, the arrangement of circular pillars (320) may be non-uniform.

### 3. Third Exemplary Microscopic Surface Pattern

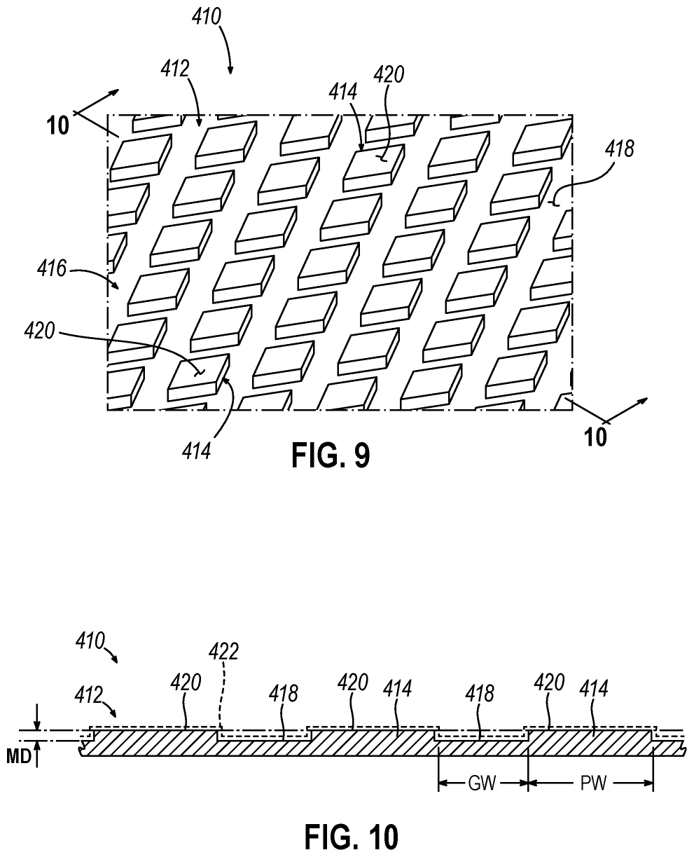

FIGS. 9-10 show a third exemplary tissue release feature (410) including a third exemplary microscopic surface pattern (412) in the form of a cross-groove pattern applied to base surface (414) ultrasonic blade (60) of FIG. 2. Particularly, FIG. 9 shows a perspective view of microscopic surface pattern (412), and FIG. 10 shows a cross-sectional view of microscopic surface pattern (412) of FIG. 9 taken along line 10-10 of FIG. 9. Particularly, microscopic surface pattern (412) of tissue release feature (410) includes a plurality of grooves (418) and a plurality of diamond shaped pillars (420). As shown, grooves (418) intersect diamond shaped pillars (420) at non-right angles. It is envisioned that grooves (418) may intersect diamond shaped pillars (420) at variety of other suitable angles. As shown in FIG. 10, grooves (418) are recessed relative to circular pillars (420). An optional hydrophobic coating (422) may be applied to tissue release feature (210) to reduce tissue sticking. As shown, hydrophobic coating (422) has a thickness that is less than the microscopic depth (MD) of grooves (418).

In some versions, the microscopic depth (MD) of grooves (418) relative to diamond shaped pillars (420) may range from between approximately 5 microns and approximately 50 microns. Diamond shaped pillars (420) may have a groove width (GW) of between approximately 20 microns and approximately 150 microns. Diamond shaped pillars (420) may have a pillar width (PW) of between approximately 20 microns and approximately 200 microns. Additionally, the arrangement of diamond shaped pillars (420) may be non-uniform.

### 4. Fourth Exemplary Microscopic Surface Pattern

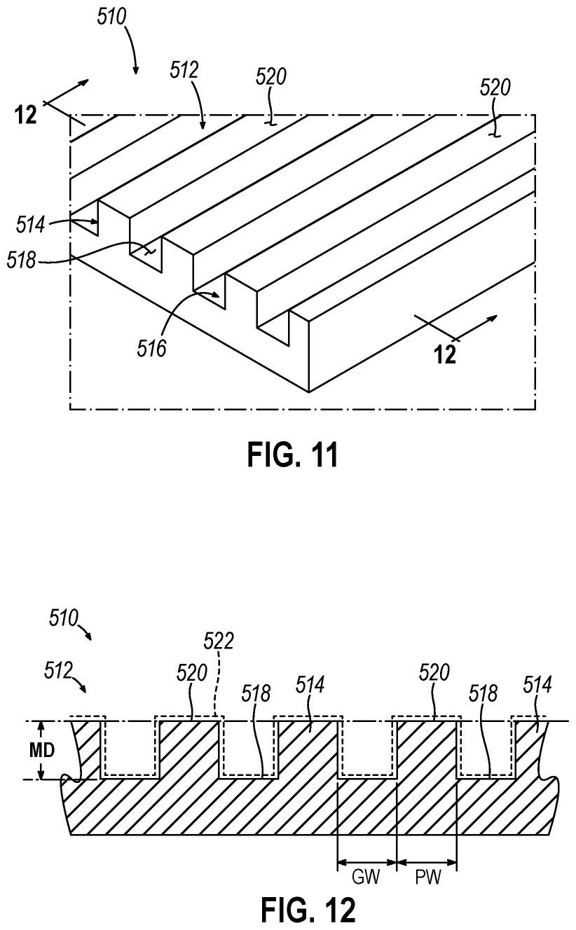

FIGS. 11-12 show a fourth exemplary tissue release feature (510) including a third exemplary microscopic surface pattern (512) in the form of a slotted pattern applied to base surface (514) ultrasonic blade (60) of FIG. 2. Particularly, FIG. 11 shows a perspective view of microscopic surface pattern (512), and FIG. 12 shows a cross-sectional view of microscopic surface pattern (512) of FIG. 11 taken along line 12-12 of FIG. 11. Particularly, microscopic surface pattern (512) of tissue release feature (510) includes a plurality of grooves (518) and a plurality of slotted pillars (520). As shown, grooves (518) are disposed parallel to slotted pillars (520). As shown in FIG. 10, grooves (518) are recessed relative to slotted pillars (520). An optional hydrophobic coating (522) may be applied to tissue release feature (510) to reduce tissue sticking. As shown, hydrophobic coating (522) has a thickness that is less than the microscopic depth (MD) of grooves (518).

In some versions, the microscopic depth (MD) of grooves (518) relative to slotted pillars (520) may range from between approximately 5 microns and approximately 50 microns. Slotted pillars (520) may have a groove width (GW) of between approximately 20 microns and approximately 150 microns. Slotted pillars (520) may have a pillar width (PW) of between approximately 20 microns and approximately 200 microns. Additionally, the arrangement of slotted pillars (520) may be non-uniform.

### 5. Fifth Exemplary Microscopic Surface Pattern

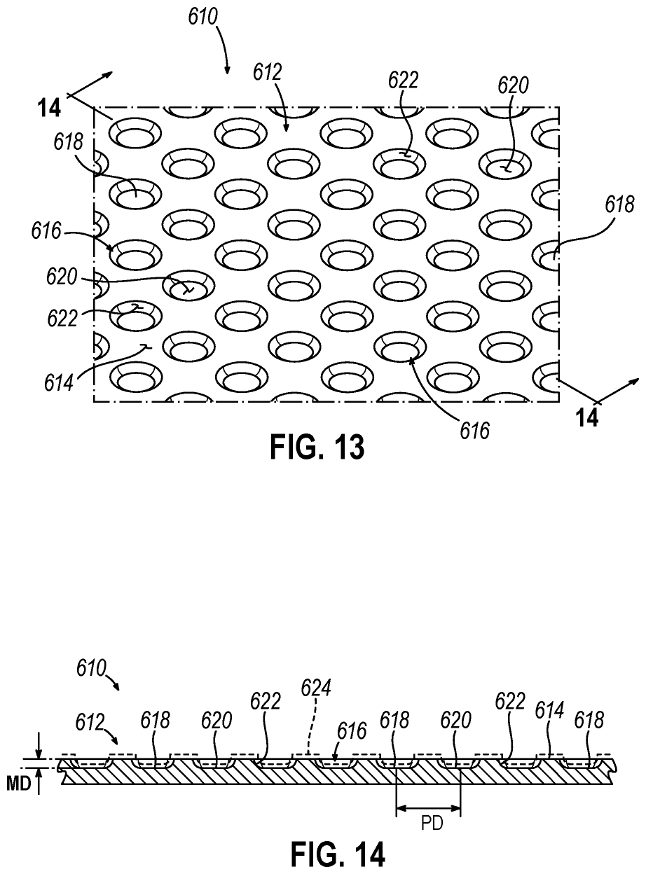

FIGS. 13-14 show a fifth exemplary tissue release feature (610) including a fifth exemplary microscopic surface pattern (612) in the form of an array of dimples arranged in a grid pattern applied to base surface (614) of ultrasonic blade (60) of FIG. 2. Particularly, FIG. 13 shows a perspective view of microscopic surface pattern (612) applied to ultrasonic blade (60) of FIG. 2, and FIG. 14 shows a cross-sectional view of microscopic surface pattern (612) of FIG. 13 taken along line 14-14 of FIG. 13. As best shown in FIG. 14, microscopic surface pattern (612) includes a plurality of recessed portions (616) that are recessed at a microscopic depth (MD) from base surface (614). Microscopic surface pattern (612) includes individual dimples (618). Dimples (618) may have a microscopic depth (MD) of approximately 5 microns to approximately 25 microns. Dimples (618) may have a diameter of between approximately 20 microns and approximately 150 microns. Dimples (618) may have a pitch distance of approximately the diameter of dimple (618) plus 1 micron to the diameter of dimple (618) plus 20 microns (i.e., between approximately 21 microns and approximately 170 microns). For example, individual dimples (618) of microscopic surface pattern (612) may have a diameter of approximately 38 microns and be spaced at a pitch of approximately 50 microns. However, other suitable diameters and spacings of dimples (618) are also envisioned.

While microscopic surface pattern (612) is shown as including individual dimples (618) arranged in discrete rows and discrete columns, microscopic surface pattern (612) may be generally non-uniform and not arranged in discrete rows and columns in a grid pattern. Dimples (618) may be hemispherical or hemispherical with a generally planar bottom (620) as shown in FIGS. 13-14. Dimples (618) may have arcuate sidewalls (622) that taper inwardly toward bottom (620).

### 6. Sixth Exemplary Microscopic Surface Pattern

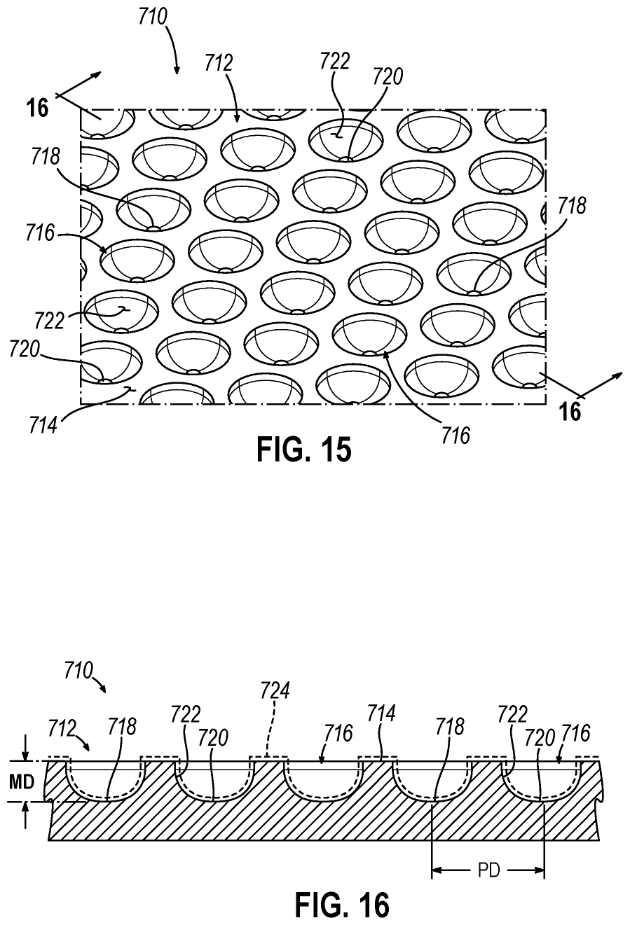

FIGS. 15-16 show a sixth exemplary tissue release feature (710) including a sixth exemplary microscopic surface pattern (712) in the form of an array of dimples arranged in a honeycomb pattern applied to base surface (714) of ultrasonic blade (70) of FIG. 2. As shown, adjacent rows of dimples (718) are offset from each other. Particularly, FIG. 15 shows a perspective view of microscopic surface pattern (712) applied to ultrasonic blade (70) of FIG. 2, and FIG. 16 shows a cross-sectional view of microscopic surface pattern (712) of FIG. 15 taken along line 16-16 of FIG. 15. As best shown in FIG. 16, microscopic surface pattern (712) includes a plurality of recessed portions (716) that are recessed at a microscopic depth (MD) from base surface (714). Microscopic surface pattern (712) includes individual dimples (718). Dimples (718) may have a microscopic depth (MD) of between approximately 5 microns and approximately 25 microns. Dimples (718) may have a diameter of between approximately 20 microns and approximately 150 microns. Dimples (718) may have a pitch distance of between approximately the diameter of dimple (718) plus 1 micron and the diameter of dimple (718) plus 20 microns (i.e., between approximately 21 microns and approximately 170 microns). However, other suitable diameters and spacings of dimples (718) are also envisioned.

While microscopic surface pattern (712) is shown as including individual dimples (718) arranged in a honeycomb pattern, microscopic surface pattern (712) may be generally non-uniform. Dimples (718) may be hemispherical or hemispherical with a generally planar bottom (720) as shown in FIGS. 15-16. Dimples (718) may have arcuate sidewalls (722) that taper inwardly toward bottom (720).

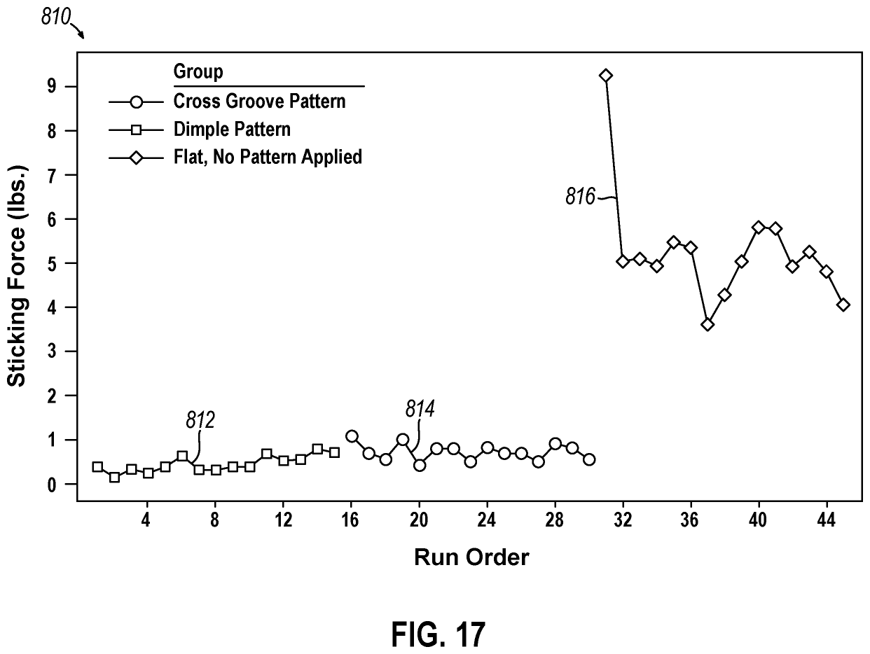

FIG. 17 shows an exemplary line graph (810) showing exemplary first, second, and third data series (812, 814, 816) pertaining to sticking force versus run order. Particularly, FIG. 17 shows a first data series (812) similar to microscopic surface pattern (212) of FIG. 6, a second data series (814) similar to microscopic surface pattern (612) of FIG. 13, and a third data series (816) for a flat non-patterned surface. For example, first, second, and third data series (812, 814, 816) may measure tissue sticking force of jejunum tissue for parallel plate RF electrodes using an accelerated sticking test method. Electrodes may be manufactured from the same flat stock of stainless steel. Third data series (816) using a flat non-patterned surface may serve as a control. First, second, and third data series (812, 814, 816) do not include hydrophobic coatings, but may optionally include hydrophobic coatings (222, 322, 422, 522, 624, 724).

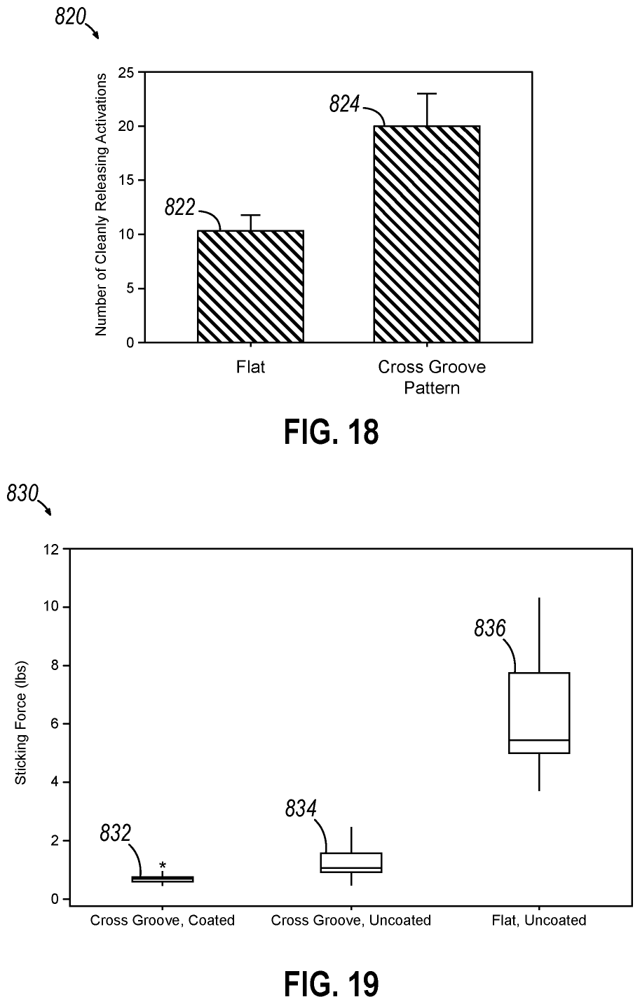

FIG. 18 shows a box plot graph (820) of exemplary first and second data series (822, 824) regarding activations resulting in tissue release. First and second data series (822, 824) may measure the number of cleaning releasing activations out of 30 activations for a bipolar tissue sealing instrument (e.g., electrosurgical instrument (110) of FIGS. 3-4), where electrodes (188, 190) are dip coated into a dispersion of room-temperature-vulcanizing (RTV) silicone. For example, first data series (822) may pertain to a dip coated flat non-patterned surface, and second data series (824) may be similar to microscopic surface pattern (232) of FIG. 6 but including a dip coating. As previously described with reference to FIGS. 5-6, rectangular pillars (220) may have a width of approximately 140 microns and a length of approximately 140 microns, which may be separated by a grid of grooves (218) having a groove width (GW) of approximately 96 microns. Grooves (218) may have a 10 micron depth. As shown in FIG. 18, first data series (822) may cleanly release ten out of thirty times, while second data series (824) may cleanly release twenty out of thirty times.

FIG. 19 shows a box plot graph (830) showing exemplary first, second, and third data series (832, 834, 836) pertaining to sticking force. Particularly, FIG. 19 shows a first data series (832) similar to microscopic surface pattern (232) of FIG. 6 and including a plasma coating, a second data series (834) similar to microscopic surface pattern (232) of FIG. 6 but omitting a plasma coating, and a third data series (836) pertaining to a non-coated flat non-patterned surface. For example, first, second, and third data series (832, 834, 836) may measure tissue sticking force of jejunum tissue for parallel plate bipolar electrodes (e.g., electrodes (188, 190)). For example, 10 W of power may be applied with a 330 kHz waveform and a compression force of 460 kPa until a 500 Ohm termination impedance is obtained. Electrodes may be manufactured from the same flat stock of stainless steel. Third data series (836) may serve as a control and have a non-coated flat non-patterned surface.

### C. Nanoscopic Surface Roughness

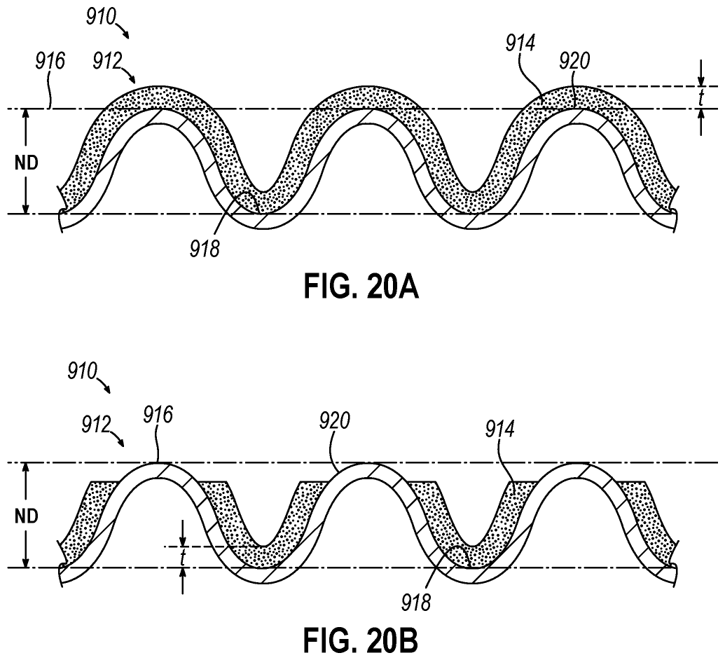

FIGS. 20A-20B show a third exemplary tissue release feature (910) that includes a nanoscopic surface roughness (912). Particularly, FIG. 20A shows a schematic cross-sectional view of nanoscopic surface roughness (912) that includes a hydrophobic coating (914) applied to a base surface (916) of ultrasonic blade (60) of FIG. 2, prior to a portion of hydrophobic coating (914) being worn away. Hydrophobic coating (914) may be applied to the energized feature of instrument (10, 110) that seals and/or cuts tissue in order to reduce tissue sticking. As shown in FIG. 20A, at the beginning of the useful life of the energized feature, hydrophobic coating (914) may completely cover base surface (916), and tissue sticking may be barely perceptible or non-existent.

At least one of valleys (918) of nanoscopic surface roughness may optionally include hydrophobic coating (914). Hydrophobic coating (914) may have a thickness (t) that is less than a nanoscopic depth (ND) of nanoscopic surface roughness (912). The nanoscopic scale (or nanoscale) may refer to a length scale applicable to nanotechnology, such as between approximately 1-100 nanometers. For example, hydrophobic coating (914) may have thickness (t) of between approximately 4 nanometers and approximately 100 nanometers, or more particularly between approximately 25 nanometers and approximately 60 nanometers, or more particularly between approximately 25 nanometers and approximately 35 nanometers. As shown, thickness (t) of hydrophobic coating (914) is generally uniform. However, non-uniform applications of hydrophobic coating (914) are also envisioned. Nanoscopic surface roughness (912) may be applied to an electrode surface with laser ablation (such as with picosecond or femtosecond lasers), chemical etching, or a similar process. For example, for a coating with a thickness of 20 nanometers, regularly spaced or irregularly spaced grooves of depths of 60 nanometers may be appropriate.

To improve the durability of these hydrophobic coatings (914), which may be on the order of several nanometers to approximately 50 nanometers or more, nanoscopic surface roughness (912) may be applied to the energized feature. As shown, nanoscopic surface roughness (912) includes a plurality of valleys (918) that are recessed at nanoscopic depth (ND) from base surface (916). The increased nanoscopic surface roughness (912) may function to increase the number of bonding sites for hydrophobic coating (914). The increased nanoscopic surface roughness (912) may also function to protect hydrophobic coating (914) from high shear forces and/or high compressive loads that may disrupt or remove hydrophobic coating (914). Depending on the geometry, the nanoscale roughness may serve to further increase the hydrophobicity of the surface beyond that of the coating alone. A textured surface with feature depths greater than thickness (t) of hydrophobic coating (914) may increase the durability of hydrophobic coating (914) by protecting hydrophobic coating (914) from high shear and compressive forces and by providing increased surface area for bonding of hydrophobic coating (914).

FIG. 20B shows a schematic cross-sectional view of nanoscopic surface roughness of FIG. 20A, after a portion of hydrophobic coating (914) is worn away. At the end of the useful life of the energized feature, tissue sticking may marginally increase due to loss of hydrophobic coating (914) on an outer surface (920) of ultrasonic blade (60). Hydrophobic coating (914), contained within valleys (918), may still provide a reduction in tissue sticking compared to a flat electrode at a similar amount of usage.

### D. Hierarchical Surface Structure

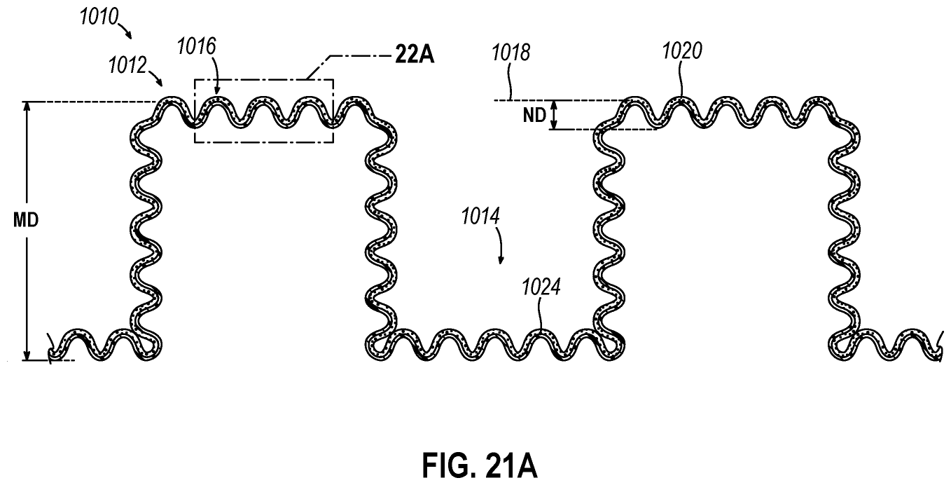

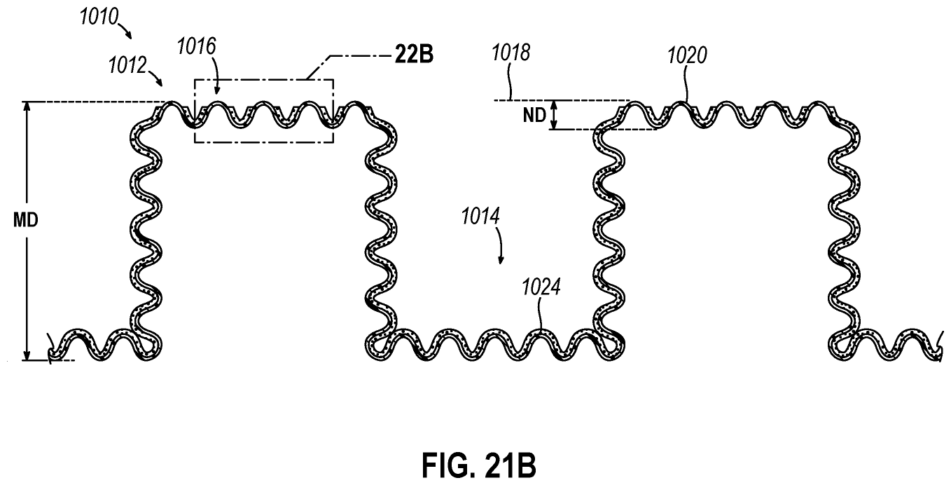

FIG. 21A shows a schematic view of a fourth exemplary tissue release feature (1010) that includes an exemplary hierarchical surface structure (1012) applied to ultrasonic blade (60) of FIG. 2. Hierarchical surface structure (1012) includes both a microscopic surface pattern (1014) and a nanoscopic surface roughness (1016). Hierarchical surface structure (1012) refers to surface roughness on the order of multiple length scales. Multiple length scales of surface features, or surface roughness, may be incorporated together to improve hydrophobicity durability and may provide a longer lasting benefit to tissue sticking compared to microscopic surface patterns (212, 312, 412, 512, 612, 712) and nanoscopic surface roughness (912) considered alone. For example, microscopic surface pattern (1014) may be similar to microscopic surface patterns (212, 312, 412, 512, 612, 712) described above with reference to FIGS. 5-14. Similarly, nanoscopic surface roughness (1016) may be similar to nanoscopic surface roughness (912) described above with reference to FIGS. 20A-20B. Hierarchical surface structure (1012) includes a base surface (1018) configured to contact tissue.

Hierarchical surface structure (1012) may increase the hydrophobicity of the surface and may improve the durability of the surface features. Improving the durability of the surface features may improve the hydrophobicity and non-stick performance. By increasing the hydrophobicity of the energized feature of instrument (10, 110), the tissue is less likely to stick to instrument (10, 110) when under high heat and pressure. By superimposing nanoscale roughness onto a surface with microscale roughness or patterns, the hydrophobicity of base surface (1018) may increase compared to base surfaces with only a single scale of roughness. Additionally, the hydrophobic and nonstick performance of the energized feature may be improved by the addition of nanoscale roughness on microscale roughness or patterns.

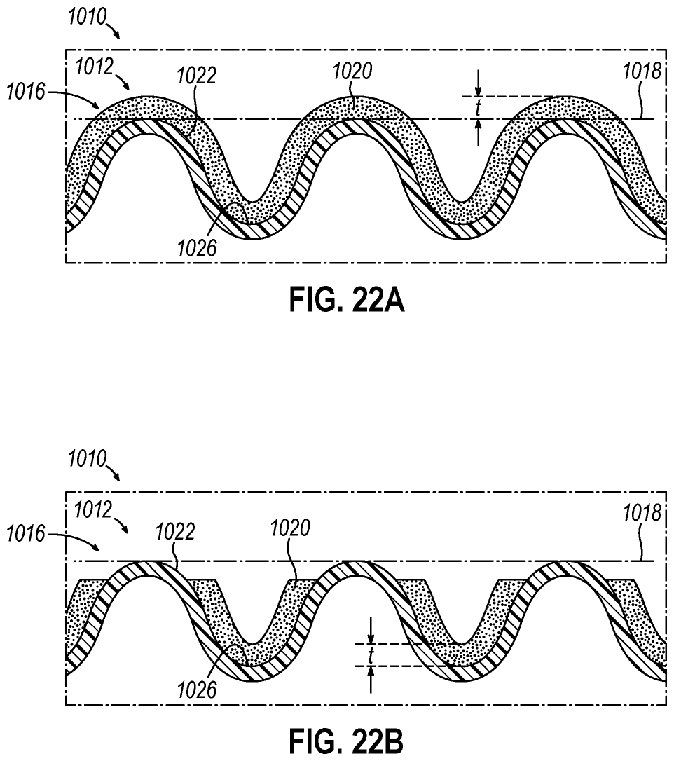

Hierarchical surface structure (1012) may optionally include a hydrophobic coating (1020). Particularly, FIG. 22A shows an enlarged cross-sectional view of hierarchical surface structure (1012) of FIG. 21A, prior to a portion of hydrophobic coating (1020) being worn away. Hydrophobic coating (1020) may be applied to an outer surface (1022) with a thickness (t) of between approximately 4 nanometers and approximately 150 nanometers. The nanoscale surface roughness (1016) provides additional surface area for hydrophobic coating (914) to bond and provides protection from high shear and compressive forces that may disrupt or cause removal of hydrophobic coating (1020) disposed on a flat surface.

Hierarchical surface structure (1012) may be applied to base surface (1018) of the energized feature using laser ablation, chemical etching, or a suitable manufacturing process. For example, laser ablation using nanosecond lasers may quickly and accurately produce microscopic surface pattern (1014) with grooves of depths of approximately 5 microns or more and with spot sizes of approximately 25 microns or greater. Picosecond or femtosecond lasers may form nanoscopic surface roughness (1016), or chemical etching may be applied as a secondary operation for producing nanoscale surface roughness (1016).

As shown in FIGS. 21A-21B, microscopic surface pattern (1014) includes a plurality of recessed portions (1024) that are recessed at a microscopic depth (MD) from base surface (1018). While microscopic surface pattern (1014) may have a minimum thickness for laser ablation applications, microscopic depth (MD) may be smaller when alternative techniques are used to form the microscopic surface pattern (1014). Nanoscopic surface roughness (912) includes a plurality of valleys (1026) that are recessed at a nanoscopic depth (ND) from base surface (1018). In some versions, nanoscopic depth (ND) may be greater than thickness (t) of hydrophobic coating (1020). For example, if an approximately 20 nanometer thick hydrophobic coating (1020) is applied, nanoscopic depth (ND) may be selected to be approximately 30-100 nanometers. FIG. 21B shows a schematic cross-sectional view of hierarchical surface structure (1012) of FIG. 20A, after a portion of a hydrophobic coating (1020) is worn away. Similarly, FIG. 22B shows an enlarged cross-sectional view of hierarchical surface structure (1012) of FIG. 21B, after a portion of hydrophobic coating (1020) is worn away. As shown, a significant portion of hydrophobic coating (1020) may be retained after use, which aids in tissue release.

### E. First Exemplary Method of Manufacturing

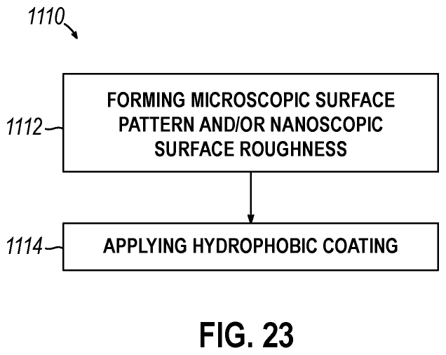

An exemplary method (1110) of manufacturing an energized feature of instrument (10, 110) is shown in FIG. 23. Energized feature includes base surface (214, 314, 414, 514, 614, 714, 916, 1018) configured to contact tissue. At step (1112), method (1110) includes using at least one manufacturing process to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature. For example, the manufacturing process may include using at least one of laser ablating or chemical etching to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature.

To form microscopic surface pattern (212, 312, 412, 512, 612, 712, 1014), a laser, such as one operating using a Yb:Fiber medium at wavelengths in the infrared region may be used to create microscopic surface pattern (212, 312, 412, 512, 612, 712, 1014). The laser may operate using nanosecond pulses (such as those between approximately 9 nanoseconds and approximately 200 nanoseconds) at an average power of approximately 20 Watts. The laser may operate with a minimum focal diameter of approximately 40 microns and a focal length of approximately 100 millimeters. The energized feature may be placed on an x, y, z stage such that microscopic surface pattern (212, 312, 412, 512, 612, 712, 1014) may be applied to the entire tissue contacting surface or microscopic surface pattern (212, 312, 412, 512, 612, 712, 1014) may be applied to only on select areas of the tissue contacting surface. To form nanoscopic surface roughness (912, 1016), a similar laser operating with femotosecond or picosecond pulses may be used to create the nanoscale roughness. Optical parameters, such as focal diameter and focal length, may be varied. While tissue release features (210, 310, 410, 510, 610, 710, 910, 1010) are described above with regard to one or more subtractive manufacturing processes that removes material from base surface (214, 314, 414, 514, 614, 714) to form recessed portions (216, 316, 416, 516, 616, 716) or valleys (918, 1026), it is also envisioned that tissue release features (210, 310, 410, 510, 610, 710, 910, 1010) may be formed using additive manufacturing, such that base surface (214, 314, 414, 514, 614, 714, 916, 1018) is built up to extend further than recessed portion (216, 316, 416, 516, 616, 716, 1024) or valleys (918, 1026).

At step (1114), method (1110) may include applying hydrophobic coating (222, 322, 422, 522, 624, 724, 914, 1020) to at least one of recessed portions (216, 316, 416, 516, 616, 716, 1024) of microscopic surface pattern (212, 312, 412, 512, 612, 712, 1014) or valleys (918, 1026) of nanoscopic surface roughness (912, 1016). For example, hydrophobic coating (222, 322, 422, 522, 624, 724, 914, 1020) may be applied to microscopic surface pattern (212, 312, 412, 512, 612, 712), nanoscopic surface roughness (912), or hierarchical surface structure (1012) that includes microscopic surface pattern (1014) and nanoscopic surface roughness (1016). For example, hydrophobic coating (222, 322, 422, 522, 624, 724, 914, 1020) may include a silicone dip coating, a low-pressure plasma coating, or self-assembled monolayers.

Various methods may be used to apply hydrophobic coating (222, 322, 422, 522, 624, 724, 914, 1020). In some versions, a silicone dip coating may be applied by dipping each individual assembled jaw containing energized features into a Room Temperature Vulcanising (RTV) silicone dispersion, with or without a heat curing (e.g., vulcanization) step. In other versions, the low-pressure plasma coating may be applied by placing the energized features into a vacuum chamber and coating the energized features using a low-pressure plasma process with a silicone compound, such as hexamethyldisiloxane or polydimethylsiloxane, and/or a fluorinated compound. This may be a batch process where multiple components are coated simultaneously. Still yet in other versions, self-assembled monolayers may be applied by dipping each individual assembled jaw containing the surface structured electrodes into a solution containing a fluorinated self-assembled monolayer. Still yet in other versions, hydrophobic coating (222, 322, 422, 522, 624, 724, 914, 1020) may include titanium nitride, chromium nitride, or titanium aluminum nitride using a physical vapor deposition (PVD) process. Optionally, after hydrophobic coating (222, 322, 422, 522, 624, 724, 914, 1020) is applied, an anti-stick phospholipid solution may be applied to the energized feature to reduce sticking during an electrosurgical procedure. The anti-stick phospholipid solution may be made from a fatty acid. Using the anti-stick phospholipid solution may help reduce the buildup of eschar on the energized feature during the electrosurgical procedure. In some versions, the anti-stick phospholipid solution may be applied after each subsequent use of the energized feature prior to the next subsequent use the energized feature.

### F. Second Exemplary Method of Manufacturing

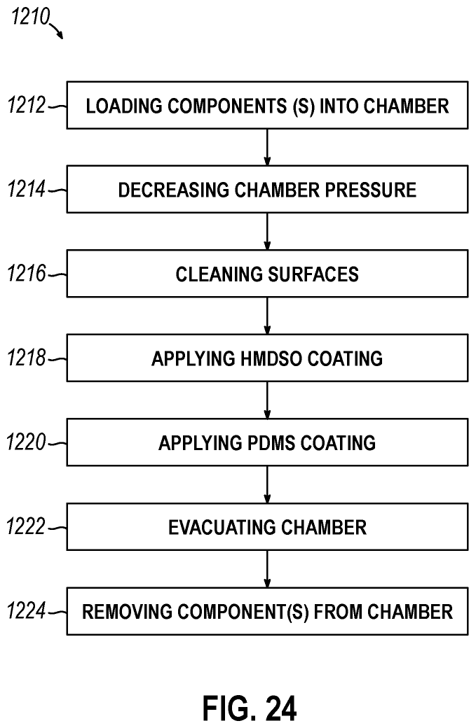

FIG. 24 shows a diagrammatic view of a second exemplary method (1210) of applying a hydrophobic coating to the energized feature of FIG. 2. As described above with reference to FIG. 23, method (1210) may include using at least one manufacturing process to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature. At step (1212), method (1210) includes loading the energized feature into a vacuum chamber. In some instances, an entire jaw or jaws may be inserted into the vacuum chamber. Once the desired component(s) are loaded into the vacuum chamber, the door may be closed, and an activation mechanism (e.g., a button of a human machine interface (HMI)) may be actuated to start the plasma cycle. This may be a batch process where multiple components are coated simultaneously. In some versions, the components may be placed on a flat tray. At step (1214), method (1210) includes decreasing the pressure of the vacuum chamber prior to applying the first coating. Step (1214) may include vacuuming out air from the vacuum chamber, to vacuum pump down the vacuum chamber. At step (1216), method (1210) includes plasma cleaning at least one surface of the energized feature after decreasing the pressure of the vacuum chamber and prior to applying the first coating. Plasma cleaning the at least one surface of the energized feature may include plasma cleaning the at least one surface of the energized feature using oxygen or argon gas.

At step (1218), method (1210) includes applying a first coating that includes hexamethyldisiloxane (HMDSO) to the energized feature. The first coating may serve as a primer layer. In some versions, the first coating may consist essentially of hexamethyldisiloxane (HMDSO). The first coating may have a thickness that ranges from between approximately 1 and approximately 10 nanometers. In some versions, the first coating to have a thickness that ranges from between approximately 1 and approximately 3 nanometers. At step (1218), method (1210) may include applying hexamethyldisiloxane (HMDSO) coating. For example, two valve hardware may be utilized.

At step (1220), method (1210) includes applying a second coating that includes polydimethylsiloxane (PDMS) to the energized feature after applying the first coating. In some versions, the first coating may consist essentially of polydimethylsiloxane (PDMS). In some versions, the second coating may have a thickness that ranges from between approximately 15 and approximately 35 nanometers. The first and second coatings may have a combined thickness that ranges from between approximately 4 and approximately 150 nanometers. In some versions, the first and second coatings have a combined thickness that ranges from between approximately 15 and approximately 60 nanometers. Liquid flow control valves, argon gas, and polymethylhydrosiloxane, trimethysilyl terminated (PMHS) may be utilized. Steps (1212, 1214, 1216, 1218, 1220) may be controlled using a machine program with a closed loop. An optional third coating may be subsequently applied. For example, the third coating may include a fluorinated monomer to the energized feature after applying the first and second coatings to the energized feature.

At step (1222), method (1210) includes evacuating the vacuum chamber after applying the second coating. Step (1222) may be controlled using an operating procedure. At step (1224), method (1210) may include removing component(s) from the vacuum chamber.

### G. Third Exemplary Method of Manufacturing

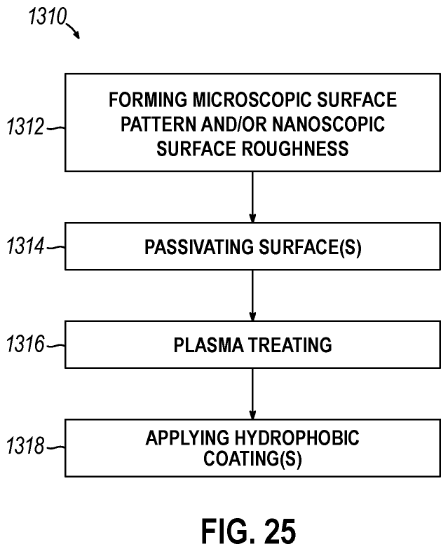

A third exemplary method (1310) of manufacturing an energized feature of instrument (10, 110) is shown with reference to FIG. 25. The energized feature includes base surface (214, 314, 414, 514, 614, 714, 916, 1018) that is configured to contact tissue. As previously described, the energized feature is intended to include at least one of ultrasonic blade (60) shown in FIGS. 1-2, electrodes (188, 190) of electrode assembly (186)) shown in FIG. 4, or another suitable energized feature.

At step (1312), method (1310) includes using at least one manufacturing process to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature (which may also be referred to as “surface structuring” the energized feature). For example, the manufacturing process(es) may include using at least one of laser ablating or chemical etching to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature. According to an exemplary embodiment, the microstructure shown and described above with reference to FIGS. 5 and 6 may be applied to the energized feature. As previously described, to form microscopic surface pattern (212, 312, 412, 512, 612, 712, 1014), a laser, such as one operating using a Yb:Fiber medium at wavelengths in the infrared region. Similarly, to form nanoscopic surface roughness (912, 1016), a similar laser operating with femotosecond or picosecond pulses may be used to create the nanoscale roughness. Additive manufacturing may be alternatively used. Optionally, in some versions, the energized feature may be placed in a water-cooled or air-cooled fixture to cool the energized feature during application of the microstructure (e.g., using the laser as described above) which may minimize variation in base surface (214, 314, 414, 514, 614, 714, 916, 1018) which may be induced by the heat of laser ablation to the tissue-contacting surfaces of the energized feature.

At step (1314), method (1310) may include passivating surface(s) of the energized feature. For example, after application of microscopic surface pattern (212, 312, 412, 512, 612, 712, 1014) and/or nanoscopic surface roughness (912, 1016) on base surface (214, 314, 414, 514, 614, 714, 916, 1018) of the energized feature, the energized feature may be placed in an acid bath (e.g., a citric acid bath or a nitric acid bath) to clean and passivate the surfaces of the energized feature. Passivating surfaces of the energized feature may be performed prior to applying one or more hydrophobic coatings (222, 322, 422, 522, 624, 724, 914, 1020).

At step (1316), method (1310) may include optionally plasma treating the energized feature. For example, the energized feature may be placed into a low-pressure plasma chamber where the energized feature undergoes plasma treatment to clean and activate surface(s) of the energized feature. The plasma treatment may remove surface contaminates (e.g., organic residues) and/or increase surface energy of the energized feature. The plasma treatment may prepare the surface to improve bond strength and coverage of the hydrophobic coating to the energized feature. In some versions, a batch process may be used where the energized feature is placed into the plasma chamber, the plasma chamber is closed and the pressure lowered to about 0.3 millibar, oxygen is introduced as the process gas, and the energized feature(s) are plasma treated for a duration about 5 minutes using a generator operating in the kilohertz frequency range. Optionally, in some versions, argon, or a mixture of argon and oxygen, may be alternatively used as the process gas. The plasma chamber may then be vented and the energized feature subsequently removed. Optionally, in some versions, atmospheric plasma treatment may be used instead of a low-pressure plasma, where the energized feature may be treated one by one instead of as a batch process within the plasma chamber.

At step (1318), method (1310) may include applying one or more hydrophobic coatings (222, 322, 422, 522, 624, 724, 914, 1020) to at least one of recessed portions (216, 316, 416, 516, 616, 716, 1024) of microscopic surface pattern (212, 312, 412, 512, 612, 712, 1014) or valleys (918, 1026) of nanoscopic surface roughness (912, 1016). For example, hydrophobic coating (222, 322, 422, 522, 624, 724, 914, 1020) may be applied to microscopic surface pattern (212, 312, 412, 512, 612, 712), nanoscopic surface roughness (912), or hierarchical surface structure (1012) that includes microscopic surface pattern (1014) and nanoscopic surface roughness (1016). In some versions, the hydrophobic coating may be applied immediately following plasma treatment, or in other versions within about one hour following plasma treatment. Applying the hydrophobic coating shortly after the plasma treatment may improve surface energy of the surface (which may increase hydrophobic coating coverage) and/or may reduce introduction of contaminants.

Various methods may be used to apply hydrophobic coating (222, 322, 422, 522, 624, 724, 914, 1020). In some versions, the hydrophobic coating may be applied using a dip coating process, where the energized feature is dip coated with a silicone solution. For example, the dip coating may be applied by dipping each individual assembled jaw containing the energized feature into the hydrophobic coating. After the hydrophobic coating is applied (e.g., using a dip coating), the energized feature may air dry. In some versions, the duration of air drying may be about 45 minutes; however, other suitable drying durations are also envisioned. After air drying, the energized feature (e.g., may be heat cured in an oven). This process may be completed on the energized feature, which is subsequently assembled into the device, or may be completed on sub-assemblies or full device assemblies. In some versions, the hydrophobic coating may also include heat curing electrodes. Heat curing may be performed at a temperature of between about 120 degrees Celsius to 200 about degrees Celsius for a duration of between about 5 minutes and about 8 hours. For example, the heat curing may be performed at a temperature of about 140 degrees Celsius for a duration of about 1 hour.

As an alternative to the hydrophobic coatings described above with reference to methods (1110, 1112) or in addition to the hydrophobic coatings described above with reference to methods (1110, 1112), an exemplary hydrophobic coating may include cross-linkable, platinum catalyst, rapid cure silicone. For example, the hydrophobic coating may include a mixture of a cross-linkable siloxane polymer and a non-cross-linkable siloxane polymer, a silicone cross-linking agent, a platinum catalyst, and one or more solvents. The hydrophobic coatings described herein may be combined with the teachings of one or more of U.S. Pat. No. 10,874,773, entitled “Two-Step Batch Process for Coating Surgical Needles,” issued Dec. 29, 2020; U.S. Pat. No. 10,589,313, entitled “Apparatus and Method for Batch Spray Coating of Surgical Needles,” issued Mar. 17, 2020; U.S. Pat. No. 10,465,094, entitled “Method of Applying Rapid Cure Silicone Lubricious Coatings,” issued Nov. 5, 2019; U.S. Pat. No. 10,441,947, entitled “Rapid Cure Silicone Lubricious Coatings,” issued Oct. 15, 2019; U.S. Pat. No. 9,434,857, entitled “Rapid Cure Silicone Lubricious Coatings,” issued Sep. 6, 2016; and U.S. Pat. No. 8,883,245, entitled “Method of Coating Surgical Needles,” issued Nov. 11, 2014, the disclosure of each of which is incorporated by reference in its entirety.

Examples and details of the cross-linkable siloxane polymer, the non-cross-linkable siloxane polymer, the silicone cross-linking agent, the platinum catalyst, and solvent(s) are shown and described in U.S. Pat. No. 9,434,857, entitled “Rapid Cure Silicone Lubricious Coatings,” issued Sep. 6, 2016, incorporated by reference above. For example, the cross-linkable siloxane polymer may have reactive functionalities or terminal functional groups, including but not limited to vinyl terminated, hydroxyl and acrylate functional groups. The cross-linkable siloxane polymers may include vinyl terminated polydialkylsiloxane or vinyl terminated polyalkoarylsiloxane. Examples include, but are not limited to, vinyl terminated siloxane polymers: polydimethyl siloxane, polydiphenylsilane-dimethylsiloxane copolymer, polyphenylmethylsiloxane, polyfluoropropylmethyl-dimethylsiloxane copolymer and polydiethylsiloxane. In TABLE 1 and TABLE 2, the cross-linkable siloxane polymer includes trimethylsilyl terminated polydimethysiloxane; however, other cross-linkable siloxane polymer described above are envisioned. For example, the non-cross-linkable siloxanes hydrophobic coating may include polydimethyl siloxane, polyalkylmethylsiloxane, such as polydiethylsiloxane, polyfluoropropylmethylsiloxane, polyoctylmethylsiloxane, polytetradecylmethylsiloxane, polyoctadecylmethylsiloxane, and polyalkylmethyl dimethylsiloxane, such as polyhexadecymethylsiloxane-dimethyl siloxane. In Table 1 and 2, the non-cross-linkable siloxane includes dimethylvinyl silyl terminated polydimethysiloxane; however, other non-cross-linkable siloxanes described above are envisioned. For example, the cross-linking agents that may be used in the coatings include conventional silicone cross-linking agents such as, for example, polymethylhydro siloxane, polymethylhydro-co-polydimethylsiloxane, polyethyhydrosiloxane, polymethylhydrosiloxane-co-octylmethylsiloxane, polymethylhydrosiloxane-co-methylphenylsiloxane. In TABLE 1 and TABLE 2, the cross-linking agent includes trimethylsilyl terminated polymethylhydrosiloxane; however, other cross-linking agents described above are envisioned. One such suitable platinum catalyst is shown and described in Example 1 of U.S. Pat. No. 9,434,857, entitled “Rapid Cure Silicone Lubricious Coatings,” issued Sep. 6, 2016, incorporated by reference above. Aromatic and aliphatic solvents may be used for the silicone dispersions. Examples of useful aromatic solvents include, but are not limited to, xylene and toluene. Aliphatic solvents include, but are not limited to, pentane, heptanes, hexane and their mixtures. For example, solvent(s) may be selected from the group consisting of xylene, toluene, pentane, hexane, heptanes, octane, Isopar K, and combinations thereof. In TABLE 1 and TABLE 2, the solvents include xylene and heptane; however, other solvents described above are envisioned. The silicone polymer components may be blended with conventional aromatic organic solvents, including, for example, xylene and aliphatic organic solvents (such as, for example, heptane or its commercial derivatives) to form coating solutions or compositions.

As an alternative to cross-linkable, platinum catalyst, rapid cure silicone coating described above, a condensation cure silicone, such as MED-4159 manufactured by NuSil®, may be applied using a dip process. Alternatively, in some versions, the hydrophobic coating may be applied as a plasma coating to the energized feature, where the energized feature is left in the plasma chamber after the plasma treatment step, and are then coated using HMDSO, PDMS, or similar coating as described above with reference to FIG. 24. Instead of a dip coating process, rapid cure silicones described above may optionally be applied using a spray application (such as by ultrasonic spray), by brushing, or by clamping the device sub-assembly or assembly onto a sponge that is saturated with the silicone. Air drying and heat cure times may remain the same or similar to dip coating. In other versions, the low-pressure plasma coating may be applied by placing the energized feature into a vacuum chamber and coating the energized feature using a low-pressure plasma process with a silicone compound. This may be a batch process where multiple components are coated simultaneously.

### i. First Example

In some versions, the hydrophobic coating may include a cross-linkable, platinum catalyst, rapid cure silicone. In some versions, a platinum cured cross linked silicone solution may be prepared using the components described below in TABLE 1.

TABLE 1
Hydrophobic Coating Formulation

| | | Weight |
|---|---|---|
| Component | Trade Name | (g) |
| Trimethylsilyl terminated | Gelest DMS T72 | 48 |
| polydimethysiloxane | | |
| Dimethylvinyl silyl terminated | Gelest DMS V52 | 48 |
| polydimethysiloxane | | |
| Platinum catalyst 0.02% solution | | 19 |
| Trimethylsilyl terminated | Gelest HMS 991 | 0.96 |
| polymethylhydrosiloxane | | |
| Solvent 1 | Xylene | 204 |
| Solvent 2 | Heptane | 746 |

A hydrophobic coating may be prepared in the following manner: 19 g of 0.02% platinum solution may be mixed with 204 g of xylene, 48 g of Gelest DMS-V52, 48 g of Gelest DMS-T72 and 0.96 g of Gelest HMS-991 using a DAC 400.1 FVZ high speed centrifugal mixer for 5 minutes at 3500 RPM. Additionally, 746 g of heptane may be added and the final mixture may be stirred using a magnetic stirrer for 2 hours. The percentage of heptane by weight in the hydrophobic coating may vary to alter the overall thickness of the coating. In some versions, the percentage of heptane by weight may be between about 60% and about 95%, while in other versions about 70% heptane by weight.

TABLE 3 shows an exemplary table of non-stick activation of one hundred sealing cycles of exemplary electrodes of a bipolar instrument using porcine jejunum tissue. An exemplary structure, plasma, and rapid cure (SPR) coating, which may be formulated using TABLE 1, may be compared to Controls 1-3. In this example, Controls 1-3 may include a condensation cure coating.

TABLE 3
Non-Stick Activations

| Example | Non-Stick Activations |
|---|---|
| Control 1 | 37 |
| Control 2 | 24 |
| Control 3 | 24 |
| SPR 1 | 69 |
| SPR 2 | 55 |
| SPR 3 | 96 |
| SPR 4 | 90 |

### ii. Second Example

In some versions, an optional hardener may be added to a cross-linkable, platinum catalyst, rapid cure silicone. A platinum cured cross linked silicone solution may be prepared using the components indicated in TABLE 2.

TABLE 2
Hydrophobic Coating Formulation

| | | Weight |
|---|---|---|
| Component | Trade Name | (g) |
| Trimethylsilyl terminated | Gelest DMS T72 | 24 |
| polydimethysiloxane | | |
| Dimethylvinyl silyl terminated | Gelest DMS V52 | 24 |
| polydimethysiloxane | | |
| Platinum catalyst 0.02% solution | | 9.5 |
| Trimethylsilyl terminated | Gelest HMS 991 | 0.48 |
| polymethylhydrosiloxane | | |
| Solvent 1 | Xylene | 102 |
| Silicone rubber base (dimethylvinyl silyl | Elkem | 160 |
| terminated polydimethysiloxane and silica | development | |
| filler) | base 44 | |
| Solvent 2 | Heptane | 1813 |

A hydrophobic coating may be prepared in the following manner: about 9.5 g of 0.02% Platinum solution may be mixed with about 102 g of xylene, about 24 g of Gelest DMS-V52, about 24 g of Gelest DMS-T72, 0.48 g of Gelest HMS-991, and 160 g of Elkem development base 44 using a FlackTek DAC 400.1 FVZ high speed centrifugal mixer for about 5 minutes at about 3500 RPM. Additionally, about 1813 g of heptane may be added and the mixture may be stirred using a magnetic stirrer for 2 hours. Air drying and heat curing steps may be similar to those described above. The dimethylvinyl silyl terminated polydimethysiloxane and silica filler may function as a hardener to increase abrasion resistance of the hydrophobic coating. The percentage of heptane by weight in the hydrophobic coating may vary the final thickness of the hydrophobic coating. In some versions, the percentage of heptane by weight may be between about 60% and about 95%, while in other versions about 70% heptane by weight.

### H. Fourth Exemplary Method of Manufacturing

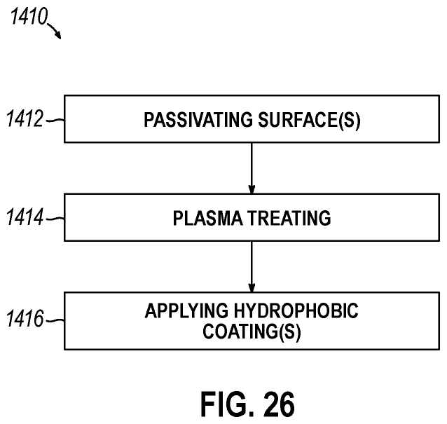

A fourth exemplary method (1410) of manufacturing an energized feature of instrument (10, 110) is shown in FIG. 26. The energized feature includes base surface (214, 314, 414, 514, 614, 714, 916, 1018) configured to contact tissue. Method (1410) is similar to method (1310) described above. However, method (1410) omits step (1312) of using at least one manufacturing process to form at least one of microscopic surface pattern (212, 312, 412, 512, 612, 712, 1014) or nanoscopic surface roughness (912, 1016) on base surface (214, 314, 414, 514, 614, 714, 916, 1018) of the energized feature. For method (1410), the passivation step (1412) similar to step (1314) may be optional. Method (1410) may include step (1414) of plasma treating the energized feature which is similar to step (1316) described above. Method (1410) may include step (1416) of applying the hydrophobic coating(s) which is similar to step (1318) described above.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of manufacturing a surgical instrument that includes an energized feature operable to apply ultrasonic energy or RF energy to tissue, the method comprising: (a) forming at least one of a microscopic surface pattern or a nanoscopic surface roughness into a base surface of the energized feature to produce at least one recessed portion; and (b) applying a hydrophobic coating that includes at least one of silicone, titanium nitride, chromium nitride, or titanium aluminum nitride to at least the recessed portion of the energized feature after forming at least one of the microscopic surface pattern or the nanoscopic surface roughness.

### Example 2

The method of Example 1, further comprising: (a) loading the energized feature into a vacuum chamber; (b) decreasing a pressure of the vacuum chamber; and (c) plasma treating the base surface and the recessed portion after decreasing the pressure of the vacuum chamber to clean and activate the energized feature.

### Example 3

The method of any one or more of Examples 1 through 2, wherein the act of plasma treating is performed prior to the act of applying the hydrophobic coating that includes silicone.

### Example 4

The method of Example 3, wherein the act of plasma treating uses at least one of oxygen or argon.

### Example 5

The method of any one or more of Examples 2 through 4, further comprising passivating the energized feature in an acid bath prior to the act of plasma treating.

### Example 6

The method of Example 1, wherein the hydrophobic coating includes at least one of titanium nitride, chromium nitride, or titanium aluminum nitride.

### Example 7

The method of any one or more of Examples 1 through 6, wherein the act of forming further comprises using at least one of laser ablating or chemical etching to form at least one of the microscopic surface pattern or the nanoscopic surface roughness.

### Example 8

The method of Example 7, wherein the at least one recessed portion is recessed at a microscopic depth from the base surface, wherein the base surface comprises a plurality of pillars, wherein the pillars include at least one of rectangular pillars, circular pillars, diamond shaped pillars, or slotted pillars.

### Example 9

The method of any one or more of Examples 1 through 8, wherein the act of applying the hydrophobic coating further comprises dipping at least the energized feature into the hydrophobic coating.

### Example 10

The method of any one or more of Examples 1 through 9, wherein the hydrophobic coating includes a cross-linkable siloxane polymer, a non-cross-linkable siloxane polymer, a silicone cross-linking agent, a platinum catalyst, and at least one solvent.

### Example 11

The method of any one or more of Examples 1 through 10, wherein the hydrophobic coating includes a silicone rubber base.

### Example 12

The method of any one or more of Examples 1 through 11, wherein the silicone rubber base includes dimethylvinyl silyl terminated polydimethysiloxane and a silica filler.

### Example 13

The method of any one or more of Examples 1 through 12, wherein the hydrophobic coating has a weight, wherein the at least one solvent includes heptane, wherein the percentage of heptane of the weight is between about 60% and about 95%.

### Example 14

The method of any one or more of Examples 1 through 13, further comprising heat curing at a temperature of between about 120 degrees Celsius to 200 about degrees Celsius after the act of applying the hydrophobic coating.

### Example 15

The method of any one or more of Examples 1 through 14, wherein the surgical instrument includes a shaft assembly and an end effector, wherein the end effector extends distally from the shaft assembly, wherein the end effector includes the energized feature, wherein the method further comprises coupling the energized feature with the end effector.

### Example 16

A method of manufacturing a surgical instrument that includes an energized feature operable to apply ultrasonic energy or RF energy to tissue, the method comprising: (a) loading the energized feature into a vacuum chamber; (b) decreasing the pressure of the vacuum chamber; (c) plasma treating at least one surface of the energized feature to clean and activate the energized feature after decreasing the pressure of the vacuum chamber; and (d) applying a hydrophobic coating that includes at least one of silicone, titanium nitride, chromium nitride, or titanium aluminum nitride after the act of plasma treating.

### Example 17

The method of Example 16, further comprising passivating the energized feature in an acid bath prior to performing the act of plasma treating.

### Example 18

The method of Example 17, wherein the acid bath includes at least one of citric acid bath or a nitric acid bath.

### Example 19

A surgical instrument comprising: (a) a shaft assembly; (b) an end effector extending distally from the shaft assembly, wherein the end effector includes an energized feature configured to apply energy to treat tissue, wherein the energized feature includes at least one of an ultrasonic blade or an electrode, the energized feature comprising: (i) a base surface configured to contact the tissue, and (ii) a recessed portion that is recessed from the base surface using at least one of a microscopic surface pattern or a nanoscopic surface roughness; and (c) a hydrophobic coating that includes at least one of silicone, titanium nitride, chromium nitride, or titanium aluminum nitride.

### Example 20

The surgical instrument of Example 19, wherein the hydrophobic coating includes a cross-linkable siloxane polymer, a non-cross-linkable siloxane polymer, a silicone cross-linking agent, a platinum catalyst, and at least one solvent.

### Example 21

A method of manufacturing a surgical instrument that includes an energized feature operable to apply ultrasonic energy or RF energy to tissue, the method comprising: (a) applying a first coating that includes hexamethyldisiloxane (HMDSO) to the energized feature; and (b) applying a second coating that includes polydimethylsiloxane (PDMS) to the energized feature after applying the first coating.

### Example 22

The method of Example 21, further comprising: (a) loading the energized feature into a vacuum chamber; and (b) decreasing the pressure of the vacuum chamber prior to applying the first coating.

### Example 23

The method of any one or more of Examples 21 through 22, further comprising plasma cleaning at least one surface of the energized feature after decreasing the pressure of the vacuum chamber and prior to applying the first coating.

### Example 24

The method of Example 23, wherein plasma cleaning the at least one surface of the energized feature further comprises plasma cleaning the at least one surface of the energized feature using oxygen or argon.

### Example 25

The method of any one or more of Examples 21 through 24, wherein applying the first coating further comprises applying the first coating to have a thickness of between 1 and 10 nanometers.

### Example 26

The method of any one or more of Examples 21 through 24, wherein applying the first coating further comprises applying the first coating to have a thickness of between 1 and 3 nanometers.

### Example 27

The method of any one or more of Examples 21 through 26, wherein applying the second coating further comprises applying the second coating to have a thickness of between 15 and 35 nanometers.

### Example 28

The method of any one or more of Examples 21 through 27, wherein the first and second coatings have a combined thickness of between 4 and 150 nanometers.

### Example 29

The method of any one or more of Examples 21 through 28, wherein the first and second coatings have a combined thickness of between 15 and 60 nanometers.

### Example 30

The method of any one or more of Examples 21 through 29, further comprising evacuating the vacuum chamber after applying the second coating.

### Example 31

The method of any one or more of Examples 21 through 30, further comprising applying a third coating that includes a fluorinated monomer to the energized feature after applying the first and second coatings to the energized feature.

### Example 32

The method of any one or more of Examples 21 through 31, further comprising using at least one manufacturing process to form at least one of a microscopic surface pattern or a nanoscopic surface roughness on the energized feature prior to applying the first coating.

### Example 33

The method of Example 32, wherein using at least one manufacturing process further comprises using at least one of laser ablating or chemical etching to form at least one of the microscopic surface pattern or the nanoscopic surface roughness on the base surface of the energized feature.

### Example 34

The method of any one or more of Examples 21 through 33, wherein the surgical instrument includes a shaft assembly and an end effector, wherein the end effector extends distally from the shaft assembly, wherein the end effector includes the energized feature.

### Example 35

The method of any one or more of Examples 21 through 34, wherein applying the first coating further comprises applying the first coating that consists essentially of the hexamethyldisiloxane (HMDSO) to the energized feature, and wherein applying the second coating further comprises applying the second coating that consists essentially of the polydimethylsiloxane (PDMS) to the energized feature after applying the first coating.

### Example 36

A method of manufacturing a surgical instrument that includes an energized feature operable to apply ultrasonic energy or RF energy to tissue, the method comprising: (a) using at least one manufacturing process to form a nanoscopic surface roughness on the energized feature; and (b) applying a hydrophobic coating to the energized feature after using at least one manufacturing process to form a nanoscopic surface roughness on the energized feature.

### Example 37

The method of Example 36, wherein applying the hydrophobic coating further comprises: (a) applying a first coating that includes hexamethyldisiloxane (HMDSO) to the energized feature; and (b) applying a second coating that includes polydimethylsiloxane (PDMS) to the energized feature after applying the first coating.

### Example 38

The method of any one or more of Examples 36 through 37, further comprising using at least one manufacturing process to form a microscopic surface roughness on the energized feature.

### Example 39

A surgical instrument comprising: (a) a shaft assembly; and (b) an end effector extending distally from the shaft assembly, wherein the end effector includes an energized feature configured to apply energy to treat tissue, wherein the energized feature includes at least one of an ultrasonic blade or an electrode, wherein the energized feature includes a surface configured to contact the tissue comprising: (i) a first coating that includes hexamethyldisiloxane (HMDSO), and (ii) a second coating that includes polydimethylsiloxane (PDMS).

### Example 40

The surgical instrument of Example 39, wherein the first and second coatings have a combined thickness of between 15 and 60 nanometers.

### Example 41

A surgical instrument comprising: (a) a shaft assembly; and (b) an end effector extending distally from the shaft assembly, wherein the end effector includes an energized feature configured to apply energy to treat tissue, wherein the energized feature includes at least one of an ultrasonic blade or an electrode, wherein the energized feature includes a base surface and a tissue release feature, the tissue release feature comprising: (i) a microscopic surface pattern comprising: (A) a plurality of recessed portions that are recessed at a microscopic depth from the base surface, and (B) a microscopic hydrophobic coating having a thickness that is less than the microscopic depth, (ii) a nanoscopic surface roughness comprising: (A) a plurality of valleys that are recessed at a nanoscopic depth from the base surface, and (B) a nanoscopic hydrophobic coating having a thickness that is less than the nanoscopic depth, or (iii) a hierarchical surface pattern comprising: (A) the recessed portions, (B) the valleys, and (C) the nanoscopic hydrophobic coating.

### Example 42

The surgical instrument of Example 41, wherein the end effector comprises a clamp arm configured to compress the tissue against the energized feature, wherein the clamp arm is pivotable toward and away from the energized feature.

### Example 43

The surgical instrument of Example 42, further comprising a waveguide, wherein the energized feature comprises the ultrasonic blade in acoustic communication with the waveguide, wherein the clamp arm is pivotable toward and away from the ultrasonic blade, wherein at least a portion of the ultrasonic blade includes the tissue release feature.

### Example 44

The surgical instrument of any one or more of Examples 41 through 43, wherein the electrode comprises an active electrode, wherein the active electrode is configured to apply radiofrequency electrosurgical energy to the tissue, wherein the active electrode includes the tissue release feature.

### Example 45

The surgical instrument of any one or more of Examples 41 through 44, wherein the energized feature comprises: (i) a first electrode, and (ii) a second electrode, wherein the first and second electrodes are configured to cooperate to apply bipolar RF energy to tissue, wherein at least one of the first and second electrodes includes the tissue release feature.

### Example 46

The surgical instrument of any one or more of Examples 41 through 45, wherein both of the first and second electrodes include the tissue release feature.

### Example 47

The surgical instrument of any one or more of Examples 41 through 46, wherein the microscopic hydrophobic coating or the nanoscopic hydrophobic coating includes at least one of a silicone compound or a fluorinated compound.

### Example 48

The surgical instrument of Example 47, wherein the silicone compound includes at least one of hexamethyldisiloxane or polydimethylsiloxane.

### Example 49

The surgical instrument of any one or more of Examples 41 through 48, wherein the nanoscopic hydrophobic coating has a thickness of between approximately 4 nanometers and approximately 150 nanometers.

### Example 50

The surgical instrument of any one or more of Examples 41 through 49, wherein the microscopic depth is between approximately 5 microns and approximately 100 microns.

### Example 51

The surgical instrument of any one or more of Examples 41 through 49, wherein the microscopic depth is between approximately 7 microns and approximately 25 microns.

### Example 52

The surgical instrument of any one or more of Examples 41 through 51, wherein the base surface comprises a plurality of pillars, wherein the recessed portion includes a plurality of grooves.

### Example 53

The surgical instrument of any one or more of Examples 41 through 52, wherein the pillars further comprise at least one of rectangular pillars, circular pillars, diamond shaped pillars, or slotted pillars.

### Example 54

The surgical instrument of any one or more of Examples 41 through 53, wherein the recessed portion is non-contiguous.

### Example 55

The surgical instrument of any one or more of Examples 41 through 54, wherein recessed portion includes a plurality of spaced dimples that are separated by the base surface.

### Example 56

The surgical instrument of Example 55, wherein the spaced dimples are arranged in a grid pattern or a honeycomb pattern.

### Example 57

A surgical instrument comprising: (a) a shaft assembly; and (b) an end effector extending distally from the shaft assembly, wherein the end effector comprises: (i) a clamp arm configured to compress tissue, and (ii) an energized feature configured to apply energy to treat tissue, wherein the energized feature includes at least one of an ultrasonic blade or an electrode, wherein the energized feature includes a base surface and a tissue release feature, wherein the tissue release feature includes a microscopic surface pattern comprising: (A) a recessed portion that is recessed at a microscopic depth from the base surface, wherein recessed portion includes a plurality of spaced dimples that are separated by the base surface.

### Example 58

The surgical instrument of Example 57, wherein the spaced dimples are arranged in a grid pattern or a honeycomb pattern.

### Example 59

A method of manufacturing a surgical instrument, wherein the surgical instrument includes a shaft assembly and an end effector, wherein the end effector extends distally from the shaft assembly, wherein the end effector includes an energized feature, wherein the energized feature is operable to apply ultrasonic energy or RF energy to tissue, wherein the energized feature includes a base surface, the method comprising: (a) forming at least one of a microscopic surface pattern or a nanoscopic surface roughness in the base surface of the energized feature; and (b) subsequently applying a hydrophobic coating to at least the energized feature.

### Example 60

The method of Example 59, wherein applying the hydrophobic coating further comprises: (a) dipping at least the energized feature into a silicone compound or a fluorinated self-assembled monomer compound, or (b) plasma coating in a low-pressure plasma chamber at least the energized feature with at least one of a silicone compound or a fluorinated compound.

### Example 61

The method of any one or more of Examples 59 through 60, wherein applying the hydrophobic coating further comprises: (a) applying a first layer of hexamethyldisiloxane to at least the energized feature, and (b) applying a second layer of polydimethylsiloxane to at least the energized feature after applying the first layer of hexamethyldisiloxane.

V. Miscellaneous

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A method of manufacturing a surgical instrument that includes an energized feature operable to apply ultrasonic energy or RF energy to tissue, the method comprising:
(a) forming at least one of a microscopic surface pattern or a nanoscopic surface roughness into a base surface of the energized feature to produce at least one recessed portion; and
(b) applying a hydrophobic coating that includes at least one of silicone, titanium nitride, chromium nitride, or titanium aluminum nitride to at least the recessed portion of the energized feature after forming at least one of the microscopic surface pattern or the nanoscopic surface roughness.

2. The method of claim 1, further comprising:
(a) loading the energized feature into a vacuum chamber;
(b) decreasing a pressure of the vacuum chamber; and
(c) plasma treating the base surface and the recessed portion after decreasing the pressure of the vacuum chamber to clean and activate the energized feature.

3. The method of claim 2, wherein the act of plasma treating is performed prior to the act of applying the hydrophobic coating that includes silicone.

4. The method of claim 3, wherein the act of plasma treating uses at least one of oxygen or argon.

5. The method of claim 2, further comprising passivating the energized feature in an acid bath prior to the act of plasma treating.

6. The method of claim 1, wherein the hydrophobic coating includes at least one of titanium nitride, chromium nitride, or titanium aluminum nitride.

7. The method of claim 1, wherein the act of forming further comprises using at least one of laser ablating or chemical etching to form at least one of the microscopic surface pattern or the nanoscopic surface roughness.

8. The method of claim 7, wherein the at least one recessed portion is recessed at a microscopic depth from the base surface, wherein the base surface comprises a plurality of pillars, wherein the pillars include at least one of rectangular pillars, circular pillars, diamond shaped pillars, or slotted pillars.

9. The method of claim 1, wherein the act of applying the hydrophobic coating further comprises dipping at least the energized feature into the hydrophobic coating.

10. The method of claim 1, wherein the hydrophobic coating includes a cross-linkable siloxane polymer, a non-cross-linkable siloxane polymer, a silicone cross-linking agent, a platinum catalyst, and at least one solvent.

11. The method of claim 10, wherein the hydrophobic coating includes a silicone rubber base.

12. The method of claim 11, wherein the silicone rubber base includes dimethylvinyl silyl terminated polydimethysiloxane and a silica filler.

13. The method of claim 12, wherein the hydrophobic coating has a weight, wherein the at least one solvent includes heptane, wherein the percentage of heptane of the weight is between about 60% and about 95%.

14. The method of claim 1, further comprising heat curing at a temperature of between about 120 degrees Celsius to 200 about degrees Celsius after the act of applying the hydrophobic coating.

15. The method of claim 1, wherein the surgical instrument includes a shaft assembly and an end effector, wherein the end effector extends distally from the shaft assembly, wherein the end effector includes the energized feature, wherein the method further comprises coupling the energized feature with the end effector.

16. A method of manufacturing a surgical instrument that includes an energized feature operable to apply ultrasonic energy or RF energy to tissue, the method comprising:
(a) loading the energized feature into a vacuum chamber;
(b) decreasing the pressure of the vacuum chamber;
(c) plasma treating at least one surface of the energized feature to clean and activate the energized feature after decreasing the pressure of the vacuum chamber; and
(d) applying a hydrophobic coating that includes at least one of silicone, titanium nitride, chromium nitride, or titanium aluminum nitride after the act of plasma treating.

17. The method of claim 16, further comprising passivating the energized feature in an acid bath prior to performing the act of plasma treating.

18. The method of claim 16, wherein the acid bath includes at least one of citric acid bath or a nitric acid bath.

19. A surgical instrument comprising:
(a) a shaft assembly;
(b) an end effector extending distally from the shaft assembly, wherein the end effector includes an energized feature configured to apply energy to treat tissue, wherein the energized feature includes at least one of an ultrasonic blade or an electrode, the energized feature comprising:
(i) a base surface configured to contact the tissue, and
(ii) a recessed portion that is recessed from the base surface using at least one of a microscopic surface pattern or a nanoscopic surface roughness; and
(c) a hydrophobic coating that includes at least one of silicone, titanium nitride, chromium nitride, or titanium aluminum nitride.

20. The surgical instrument of claim 19, wherein the hydrophobic coating includes a cross-linkable siloxane polymer, a non-cross-linkable siloxane polymer, a silicone cross-linking agent, a platinum catalyst, and at least one solvent.
